(19) United States
(12) Patent Application Publication
(10) Pub. No.: US 2017/0290772 A1
(43) Pub. Date: Oct. 12, 2017

(54) STABLE MICELLE AND/OR LIPOSOME COMPOSITIONS AND USES THEREOF

(71) Applicant: VIVACELLE BIO, INC., Chicago, IL (US)

(72) Inventor: Cuthbert SIMPKINS, Shreveport, LA (US)

(21) Appl. No.: 15/511,996

(22) PCT Filed: Sep. 16, 2015
(86) PCT No.: PCT/US15/50520

§ 371 Date: Mar. 16, 2017

Related U.S. Application Data
(60) Provisional application No. 62/051,688, filed on Sep. 17, 2014.

(51) Int. Cl. A61K 9/127; A61K 9/50; A61K 9/16

(52) U.S. Cl. CPC A61K 9/127; A61K 9/16; A61K 9/1271; A61K 9/1617; A61K 9/5084; A61K 47/48815

(57) ABSTRACT

Pharmaceutical compositions comprising stabilized self-assembling amphiphilic molecular structure (SAMS) having average diameters of 120 nm or less with a low zeta potential of less than 30 mV to be used for Total Parenteral Nutrition (TPN) or Total Parenteral Alimentation (TPA), increase blood pressure, renal dialysis, transport and release hydrophobic gases, exchange transfusion to replace blood, extracorporeal membrane oxygenation (ECMO), open constricted or blocked blood vessels, treat septic shock and irreversible shock due to sepsis and/or severe blood loss irreversible septic shock, treat dilutional coagulopathy of severe blood loss and bleeding with loss of clotting factors, and replace lost volume in childhood diseases that cause hypovolemia or severe hypovolemia, treat traumatic brain injury, burns, diagnosis of diseases and methods of making and using said compositions.

### CROSS-REFERENCE TO RELATED APPLICATIONS

This application claims the benefit of U.S. Provisional Patent Application No. 62/051,688, filed on Sep. 17, 2014, the contents of which are herein fully incorporated by reference.

### TECHNICAL FIELD

The technical field includes medical treatment, in particular, compositions for providing hyperalimentation, which is also known as total parenteral alimentation and total parenteral nutrition. In addition, compositions may include those that increase blood pressure, renal dialysis, bind to, transport and release hydrophobic gases, including but not limited to, oxygen, nitric oxide, hydrogen oxide, xenon, argon or carbon monoxide. The technical field also encompasses medical treatment, in particular, compositions for providing exchange transfusion to replace blood, a circulating medium in patients on extracorporeal membrane oxygenation (ECMO), open constricted or blocked blood vessels, treatment of septic shock, irreversible shock due to sepsis, severe blood loss due to irreversible septic shock, treatment for the dilutional coagulopathy of severe blood loss and bleeding with loss of clotting factors, and the replacement of lost blood volume in childhood diseases that cause hypovolemia/severe hypovolemia. Examples of hypovolemia include Dengue Fever and gastroenteritis. Furthermore, the technical field includes methods of making and using said compositions.

The technical field further encompasses medical treatment and, in particular, compositions to treat traumatic brain injury, burns, diagnosis of diseases and methods of making and using said compositions.

In particular, the present disclosure relates to stabilized self-assembling amphiphilic molecular structures (SAMS), such as micelles and/or liposomes, wherein the compositions comprise SAMS having an average diameter of about 90-120 nm, composed of 2-20% egg phospholipid, 10-80% soybean oil and suspended in an aqueous environment that may contain for example, NaCl (102 mM), Na (L) lactate (35 mM), L-histidine (0.1 mM). Additionally, the present disclosure relates to methods of administering said stabilized SAMS compositions to a human or animal subject.

### BACKGROUND

Lipid micelles and/or liposomes are generally unstable in non-lipid aqueous solutions, for example, when comprised as part of an emulsion. An emulsion comprises a mixture of two or more liquids that are normally immiscible with each other. One or more liquids, denoted as the dispersed phase, is dispersed in the other, which is denoted as the continuous phase. An example of an emulsion is an oil-in-water emulsion, and this is where the oil is the dispersed phase and water is the continuous phase. As mentioned above, micelles and/or liposomes are unstable in an emulsion, and this unstability can result in the micelles coalescing and becoming larger in diameter. This is critical for in vivo applications because these larger, coalesced micelles may occlude blood flow in capillaries, which can exacerbate or lead to numerous maladies.

While others in the past have attempted to make lipid micelles and/or liposomes with up to 2% egg phospholipid or egg lecithin as an emulsifier; these formulations were unable to achieve emulsion droplet size (i.e., micelle size) of less than 250 nm. (J. Pharm. Pharmacol. 42, 513-515, 1990, which is incorporated by reference in its entirety.) In fact, a decrease in the emulsion droplet size plateaued at approximately 1.2% egg phospholipid, and adding more egg phospholipid failed to decrease the emulsion droplet size further. Moreover, when lipid content was raised to above 10% of the emulsion, the size of the emulsion droplets increased up to 450 nm, even with 1.2% egg phospholipid present. Thus, there exists a need for improved formulations that provide smaller, more stable micelles/liposomes, comprising egg phospholipid as an emulsifier.

### SUMMARY

One aspect of the present disclosure relates to a pharmaceutical composition for Total Parenteral Nutrition or Total Parenteral Alimentation (TPN/TPA). The composition comprises water as a pharmaceutically acceptable carrier, a lipid component in an amount of 5-70% (w/v) of the pharmaceutical composition, and an amphiphilic emulsifier in an amount of 6% (w/v) or more of the pharmaceutical composition, wherein the lipid component and the amphiphilic emulsifier form free-moving SAMS in the water carrier, wherein the SAMS have diameters of 120 nm or less, and wherein the pharmaceutical composition is free of hemoglobin and fluorocarbon.

In some embodiments, the lipid component is present in an amount of 20-30% (w/v) of the pharmaceutical composition. In some related embodiments, the lipid component consists of soybean oil. In some related embodiments, the lipid component consists of one or more oils, or other hydrophobic fluid, with high omega 3 fatty acids oil content. In some embodiments, the lipid component consists of one or more oils high levels of docosahexaenoic acid and eicosapentaenoic acid. In some embodiments, the lipid component comprises algae oil or chia bean oil. In some other embodiments, the lipid component comprises algae oil or chia bean oil in an amount of 20-30% (w/v) of the pharmaceutical composition.

In some embodiments, the amphiphilic emulsifier is egg phospholipid. In some related embodiments, the egg phospholipid is present in an amount of 6% (w/v) or greater of the pharmaceutical composition. In some related embodiments, the egg phospholipid is present in an amount of 12% (w/v) of the pharmaceutical composition.

In some embodiments, the SAMS have diameters of 120 nm or less. In other embodiments, the SAMS have diameters of 80 nm or less, 60 nm or less, 40 nm or less, 30 nm or less, or 20 nm or less. In some embodiments, the SAMS have diameters in the range of 1-120 nm, 1-100 nm, 1-80 nm, 1-60 nm, 1-40 nm, 1-30 nm, 1-20 nm, 20-120 nm, 20-100 nm, 20-80 nm, 20-60 nm, 20-40 nm, 20-30 nm, 30-120 nm, 30-100 nm, 30-80 nm, 30-60 nm, 30-40 nm, 40-120 nm, 40-100 nm, 40-80 nm, 40-60 nm, 60-120 nm, 60-100 nm, 60-80 nm, 80-120 nm, 80-100 nm or 100-120 nm. The use of SAMS with a diameter of 120 nm or less allows the passage of the SAMS through the sinusoids of the liver much more readily than larger SAMS. This would eliminate the congestion seen with larger SAMS. Further, because the SAMS of the present disclosure pass more readily through the liver sinusoids, there is no reason to limit the percentage of the total body caloric intake that comes from them. This eliminates the need for high glucose. Without the need for a high glucose the TPN/TPA can be administered via a peripheral vein. This would greatly reduce the difficulty, discomfort and danger of giving TPN/TPA.

In some embodiments, the pharmaceutical composition further comprises a carbohydrate component in the amount of 1-25% (w/v) of the pharmaceutical composition. In some embodiments, the carbohydrate component comprises glucose. In other embodiments, the carbohydrate component consists of glucose.

In some embodiments, the pharmaceutical composition further comprises an amino acid component in a final concentration of approximately 1 nM to 100 mM. In some embodiments, the amino acid component comprises nine essential amino acids (i.e., histidine, isoleucine, leucine, lysine, methionine, phenylalanine, threonine, tryptophan, and valine). In some other embodiments, the amino acid component further comprises alanine, arginine, glycine, proline, serine, and tyrosine. In some other embodiments, the amino acid component further comprises glutamate and aspartate. In some other embodiments, the amino acid component further comprises one or more amino acids selected from the group consisting of cysteine, acetyl-cysteine, cysteine HCL, taurine, glycyl-glutamine, glycyl-tyrosine and alanyl-glutamine.

In some embodiments, the pharmaceutical composition further comprises L-histidine or histidine containing peptides at a concentration of 1 fM-100 mM. In some embodiments, the pharmaceutical composition further comprises L-histidine at a concentration of 0.1-20 mM. In some embodiments, the pharmaceutical composition further comprises L-histidine at a concentration of about 0.1 mM. In some embodiments, the pharmaceutical composition further comprises L-histidine at a concentration of about 1 mM. In some embodiments, the pharmaceutical composition further comprises L-histidine at a concentration of about 10 mM. In some embodiments, the pharmaceutical composition further comprises L-histidine at a concentration of about 100 mM.

In some embodiments, the pharmaceutical composition further comprises a buffering agent. In some embodiments, the buffering agent is selected from the group consisting of histidine, histidine-containing peptides, HEPES, TRIS and sodium bicarbonate.

In some embodiments, the pharmaceutical composition further comprises an electrolyte. In some embodiments, the electrolyte is selected from the group consisting of sodium salts, potassium salts, calcium salts, phosphorus salts and magnesium salts.

In some embodiments, the pharmaceutical composition further comprises one or more vitamins selected from the group consisting of vitamin A, vitamin D, vitamin E, vitamin C, vitamin B1 (thiamine), vitamin B2 (riboflavin), vitamin B6 (pyridoxine vitamin B12 (cyanocobalamin), vitamin K, folic acid, niacin, biotin and panothenic acid.

In some embodiments, the pharmaceutical composition further comprises one or more therapeutic agents that are compatible with TPN/TPA. In some embodiments, the one or more therapeutic agents is selected from the group consisting of ranitidine, famotidine, insulin and antibiotics.

Another aspect of the present disclosure relates to the use of small SAMS made with egg phospholipid or egg lecithin beyond 2% (w/v) including in an amount of 6% (w/v) or higher results to raise the blood pressure of a human or animal subject.

In some embodiments, pharmaceutical compositions comprising smaller and more stable micelle and/or liposome (average diameter of 1-120 nm), NaCl (102 mM), Na (L) lactate (35 mM), L-histidine (50 mM or less) made with egg phospholipid or egg lecithin beyond 2% (w/v) including in an amount of 6% (w/v) or higher are used to increase the blood pressure higher when infused after hemorrhagic shock or conditions related to lack of blood volume supply, i.e., hypovolemia, in comparison to pharmaceutical compositions comprising larger SAMS of average diameter of about 300 nm.

Another aspect of the present disclosure relates to the use of the pharmaceutical compositions comprising smaller and more stable SAMS (average diameter of 1-120 nm), NaCl (102 mM), Na (L) lactate (35 mM), L-histidine (50 mM or less) made with egg phospholipid or egg lecithin beyond 2% (w/v) including in an amount of 6% (w/v) or higher are used in renal dialysis. The stability of the SAMS allows them to be mixed with electrolytes and glucose in dialysis fluid for 6 months or more. The SAMS can keep blood pressure up and prevent hypotension during dialysis.

Another aspect of the present disclosure relates to the use of the pharmaceutical compositions comprising smaller and more stable SAMS (average diameter of 1-120 nm), NaCl (102 mM), Na (L) lactate (35 mM), L-histidine (50 mM or less) made with egg phospholipid or egg lecithin beyond 2% (w/v) including in an amount of 6% (w/v) or higher to carry hydrophobic gases such as oxygen, nitric oxide, hydrogen oxide, xenon, argon or carbon monoxide. These gases may be dissolved in a hydrophobic substance such as soybean oil or they may exist in the hydrophobic core of the SAMS as a gas alone. Each of these gases may have therapeutic effects.

Another aspect of the present disclosure relates to the use of the pharmaceutical compositions comprising smaller and more stable SAMS (average diameter of 1-120 nm nm), NaCl (102 mM), Na (L) lactate (35 mM), L-histidine (50 mM or less) made with egg phospholipid or egg lecithin beyond 2% (w/v) including in an amount of 6% (w/v) or higher in exchange transfusion to replace plasma or whole blood that contains harmful mediators, prions, viruses, bacteria, fungi, chemical or biological agents of warfare, cancer cells or other bloodstream born agents that adversely affect health. Said stable SAMS may also be loaded with anti-inflammatory oils or other anti-inflammatory substances to address an inflammatory state of the vasculature or of specific organs such as the brain, heart, intestine or kidney. The stability of the SAMS will increase the time it is resident in the intravascular space. Moreover an exchange of plasma and or whole blood can be used to provide a more receptive environment for organ transplantation free of the humoral and cellular agents of immunity. After that, transplantation inhibitors of the immune reaction could be infused. This would then be followed by the restoration of blood into the blood stream to perfuse organs that are much more resistant to immunological attack.

Another aspect of the present disclosure relates to the use of the pharmaceutical compositions comprising smaller and more stable SAMS (average diameter of 1-120 nm), NaCl (102 mM), Na (L) lactate (35 mM), L-histidine (50 mM or less) made with egg phospholipid or egg lecithin beyond 2% (w/v) including in an amount of 6% (w/v) or higher to provide a circulating medium in patients on extracorporeal membrane oxygenation (ECMO). While passing through the ECMO circuit, the more stable micelles can be loaded with oxygen that will be used by the tissues.

Another aspect of the present disclosure relates to loading the more stable SAMS of the pharmaceutical compositions comprising smaller and more stable SAMS (average diameter of 90-120 nm nm), NaCl (102 mM), Na (L) lactate (35 mM), L-histidine (10 mM or less) made with egg phospholipid or egg lecithin beyond 2% (w/v) including in an amount of 6% (w/v) or higher with nitric oxide to be utilized to open constricted or blocked blood vessels in for example, the heart, lungs, brain or legs.

Another aspect of the present disclosure relates to the use of the pharmaceutical compositions comprising smaller and more stable SAMS (average diameter of 90-120 nm), NaCl (102 mM), Na (L) lactate (35 mM), L-histidine (1 mM or less) made with egg phospholipid or egg lecithin beyond 2% (w/v) including in an amount of 6% (w/v) or higher to treat septic shock and irreversible shock due to sepsis and/or severe blood loss, or to activate clotting mechanisms.

Another aspect of the present disclosure relates to the use of the pharmaceutical compositions comprising smaller and more stable SAMS (average diameter of 90-120 nm), NaCl (102 mM), Na (L) lactate (35 mM), L-histidine (1 mM or less) made with egg phospholipid or egg lecithin beyond 2% (w/v) including in an amount of 6% (w/v) or higher to treat burns.

Another aspect of the present disclosure relates to the use of the pharmaceutical compositions comprising smaller and more stable SAMS (average diameter of 90-120 nm), NaCl (102 mM), Na (L) lactate (35 mM), L-histidine (1 mM or less) made with egg phospholipid or egg lecithin beyond 2% (w/v) including in an amount of 6% (w/v) or higher to treat dilutional coagulopathy of severe blood loss. That is, the SAMS may be combined with plasma to produce fluid that has the volume replacement and nitric oxide absorbing capability to treat bleeding and replace clotting factors lost as a result of severe blood loss.

Another aspect of the present disclosure relates to the use of the pharmaceutical compositions comprising smaller and more stable SAMS (average diameter of 90-120 nm), NaCl (102 mM), Na (L) lactate (35 mM), L-histidine (1 mM or less) made with egg phospholipid or egg lecithin beyond 2% (w/v) including in an amount of 6% (w/v) or higher to replace lost volume in childhood diseases that cause severe hypovolemia, i.e., to replace fluid leakage out of the intravascular space in patients suffering from these diseases. There are numerous diseases that occur in childhood that can cause hypovolemia or severe hypovolemia through the loss of body fluids through vomiting or diarrhea, perspiration. Examples of such childhood diseases are gastroenteritis and dengue fever.

Another aspect of the present disclosure relates to the use of the pharmaceutical compositions comprising smaller and more stable SAMS (average diameter of 90-120 nm), NaCl (102 mM), Na (L) lactate (35 mM), L-histidine (10 mM or less) made with egg phospholipid or egg lecithin beyond 2% (w/v) including in an amount of 6% (w/v) or higher to treat traumatic brain injury.

Another aspect of the present disclosure relates to a method for preparing the pharmaceutical compositions of the present disclosure. The method comprises the steps of combining water, a lipid component and an emulsifier component to form a liquid mixture having 30-80% water, 5-60% (w/v) lipid component and 6% (w/v) of more emulsifier component; homogenizing the liquid mixture under conditions that produce SAMS in the size range of 0.1-120 nm.

Another aspect of the present disclosure relates to a method for providing TPN/TPA to a subject. The method comprises the steps of administering TPN/TPA compositions of the present disclosure to a subject in need thereof an effective amount of the compositions of the present disclosure. In some embodiments, the TPN/TPA compositions of the present disclosure are administered intravenously through a central venous catheter. In other embodiments, the TPN/TPA compositions of the present disclosure are administered through a peripheral vein. The amount and infusion rate of the compositions are calculated based on the patient's need. The contents of the TPN/TPA compositions may be adjusted to meet the special need of each patient, while maintaining the desired osmolarity and daily caloric requirement of the patient.

### BRIEF DESCRIPTION OF THE DRAWINGS

FIG. 1 shows an illustration of a SAMS containing a lipophilic core and encapsulated by an emulsifier which may be a phospholipid or another amphiphilic molecule. The polar liquid carrier surrounds the SAMS. These components together create an emulsion. The polar liquid may be water. This emulsion may be formed by adding energy to the mixture by sonication, using a homogenizer or a microfluidizer.

FIG. 2 shows the relationship between SAMS size and the amount of egg phospholipid used for emulsification in exemplary embodiments. The Y axis is average SAMS diameter in nanometers. The X axis is the amount of egg phospholipid (w/v) in the emulsion.

FIG. 3 shows a summary of the results of SAMS stability studies of exemplary embodiments.

FIG. 4 shows the blood pressure response to an exemplary embodiment.

FIG. 5 shows the blood pressure response of an exemplary embodiment.

FIG. 6 shows the blood pressure response of an exemplary embodiment.

FIG. 7 shows the blood pressure response of an exemplary embodiment.

FIG. 8 shows the blood pressure response of an exemplary embodiment.

FIG. 9 shows the results of exchange transfusion to replace plasma or whole blood that may contain harmful mediators, prions, viruses, bacteria, fungi, chemical or biological agents of warfare, cancer cells or other bloodstream born agents that adversely affect health for an exemplary embodiment.

### DETAILED DESCRIPTION

The present disclosure relates to self-assembling amphiphilic molecular structures (SAMS), such as micelles and liposomes, which can be used to treat a number of different diseases.

The SAMS of the present disclosure comprises egg phospholipids at greater than 2% (w/v), and have diameters of 120 nm or less. Furthermore, SAMS are unexpectedly stable given their low zeta potential. For example, the pharmaceutical compositions of the present disclosure provide the ability to form a stable suspension of lipid SAMS, such that the composition may be stored at room temperature or under refrigeration for an extended period of time (i.e., at least 6 months).

In addition, the stable, small SAMS of the present disclosure can pass more easily through the liver and splenic sinusoids, which can reduce the adverse effects of SAMS being sequestered in the sinusoids of subjects. The enhanced properties of the lipid SAMS disclosed herein allows for said SAMS to be utilized in a wide variety of medical conditions. For instance, SAMS of the present disclosure can be used to increase blood pressure within a subject.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

As used herein, the term “amphiphilic” is a term describing a chemical compound possessing both hydrophilic (water-loving, polar) and lipophilic (fat-loving) properties.

As used herein, a composition is “free of hemoglobin, derivatives of hemoglobin, perfluorocarbon and derivatives of perfluorocarbon” if the composition does not contain any hemoglobin, derivatives of hemoglobin, perfluorocarbon and derivatives of perfluorocarbon, or if the composition contains hemoglobin, derivatives of hemoglobin, perfluorocarbon and derivatives of perfluorocarbon at levels below 0.1% w/v.

As used herein, the term “derivatives of hemoglobin” refers to fragments of hemoglobin that are capable of carrying oxygen, structurally modified hemoglobin such as hemoglobin molecules which were inter-molecularly coupled for the creation of hemoglobin polymer forms or hemoglobin molecules coupled to ligands, such as for example polyethylene glycol (PEG; cf. U.S. Pat. No. 5,478,806, which is incorporated by reference in its entirety) or hydroxy ethyl starch (HES; cf. WO 98/01158, which is incorporated by reference in its entirety) and/or to other polymers. The intermolecular linking can occur before, simultaneously with or after the intramolecular coupling. On binding to a ligand the intramolecular linking ensues by reaction with the globin chains bound to the ligand. The hemoglobin which can be used for the production of derivatives can be of human, animal, or recombinant origin, in the art, various processes are described for the production of recombinant hemoglobin, for example, expression in bacterial, yeast, or animal cells, as well as in transgene plants or animals.

As used herein, the term “derivatives of perfluorocarbon” refers to perfluorocarbons with some functional group attached, such as perfluorooctanesulfonic acid.

As used herein, the term “histidine-containing peptides” refers to peptides of 2-50 amino acid residues, preferably 2-15 amino acid residues, with a histidine content of 10% or higher (e.g., a 50 amino acid histidine-containing peptide would contain at least 5 histidine residues).

As used herein, the term “lipid” refers to a fat-soluble material that is naturally occurring, or non-naturally occurring, such a synthetic fat-soluble molecule.

As used herein, the term “self-assembling amphiphilic molecular structure(s) (SAMS)” refers to any structure or particle comprised of amphiphilic molecules that form structures in which spontaneous organization occurs with or without the introduction of energy into the system so that the polar segments spontaneously align with polar segments and the non-polar segments align with non-polar segments to form spatially separated zones that are mostly polar or non-polar. Examples include, but are not limited to, liposomes and micelles.

### 2. Pharmaceutical Compositions

Provided herein are pharmaceutical compositions. The pharmaceutical compositions can comprise an amphiphilic emulsifier, a lipid component, a carbohydrate component, an amino acid component, a buffering agent, an electrolyte, a vitamin, an antioxidant, other therapeutic agents, other components, and combinations thereof. The pharmaceutical compositions can comprise a carrier, wherein water is a preferred carrier.

The pharmaceutical compositions can comprise self-assembling amphiphilic molecular structures (SAMS). The amphiphilic emulsifier, the lipid component, the carbohydrate component, the amino acid component, the buffering agent, the electrolyte, the vitamin, the antioxidant, other therapeutic agents, other components, and combinations thereof can form SAMS and/or aid in the formation of the SAMS. For example, in some embodiments, the lipid component and the amphiphilic emulsifier can form SAMS in a water carrier.

a. Amphiphilic Emulsifier

The pharmaceutical composition can comprise an amphiphilic emulsifier. The amphiphilic emulsifier can be any amphiphile or amphiphilic molecule that is capable of forming single, double or multi-layer layer SAMS in water. Examples of amphiphilic emulsifiers include, but are not limited to, phospholipids, glycolipids, sterols, fatty acids, bile acids, saponins, amphiphilic peptides, hydrocarbon based surfactants such as sodium dodecyl sulfate (anionic), benzalkonium chloride (cationic), cocamidopropyl betaine (zwitterionic) and long chain alcohol (non-ionic).

Examples of phospholipids include natural or synthetic phospholipids such as phosphatidylcholine, phosphatidylethanolamine, phosphatidylserine, phosphatidic acid, phosphatidylglycerol, phosphatidylinositol, lisophosphatidylcholine, sphingomyelin, egg phospholipids, egg yolk lecithin, soybean lecithin, and a hydrogenated phospholipid.

Examples of the glycolipids include glyceroglycolipids and sphingoglycolipids. Examples of glyceroglycolipids include digalactosyl diglycerides (such as digalactosyl dilauroyl glyceride, digalactosyl dimyristoyl glyceride, digalactosyl dipalmitoyl glyceride, and digalactosyl distearoyl glyceride) and galactosyl diglycerides (such as galactosyl dilauroyl glyceride, galactosyl dimyristoyl glyceride, galactosyl dipalmitoyl glyceride, and galactosyl distearoyl glyceride). Examples of sphingoglycolipids include galactosyl cerebroside, lactosyl cerebroside, and ganglioside.

Examples of the sterols include cholesterol, cholesterol hemisuccinate, 3 β-[N—(N′,N′-dimethylaminoethane)carbamoyl]cholesterol, ergosterol, and lanosterol.

In some embodiments, the amphiphilic emulsifier is egg phospholipids.

In some embodiments, the emulsifier is soybean lecithin or alpha-phosphatidylcholine.

Amphiphilic peptide molecules typically have three regions: a hydrophobic tail, a region of beta-sheet forming amino acids, and a peptide epitope designed to allow solubility of the molecule in water.

In some embodiments, the amphiphilic emulsifier is a non-lipid amphiphilic emulsifier, such as amphiphilic peptides and hydrocarbon based surfactants.

In some embodiments, the pharmaceutical composition comprises the amphiphilic emulsifier in an amount of about 0.1-50% (w/v) of the pharmaceutical composition. In certain embodiments, the pharmaceutical composition comprises the amphiphilic emulsifier in an amount of about 0.1-0.5%, about 0.1-2%, about 0.1-5%, about 0.1-10%, about 0.1-15%, about 0.1-20%, about 0.1-30%, about 0.1-40%, about 0.5-2%, about 0.5-5%, about 0.5-10%, about 0.5-15%, about 0.5-20%, about 0.5-30%, about 0.5-40%, about 0.5-50%, about 2-5%, about 2-10%, about 2-15%, about 2-20%, about 2-30%, about 2-40%, about 2-50%, about 5-10%, about 5-15%, about 5-20, about 5-30%, about 5-40%, about 5-50%, about 10-15%, about 10-20%, about 10-30%, about 10-40%, about 10-50%, about 15-20%, about 15-30%, about 15-40%, about 15-50%, about 20-30%, about 20-40%, about 20-50%, about 30-40%, about 30-50% or about 40-50%, (w/v) of the pharmaceutical composition.

In certain embodiments, the pharmaceutical compositions comprises the emulsifier in an amount of about 0.5%, about 0.75%, about 1%, about 1.2%, about 1.5%, about 1.75%, about 2%, about 2.25%, about 2.5%, about 2.75%, about 3%, about 3.25%, about 3.5%, about 3.75%, about 4%, about 5%, about 6%, about 8%, about 10%, about 12%, about 14%, about 16%, about 18%, about 20%, about 25%, about 30%, about 35%, about 40%, about 45% or about 50% (w/v) of the pharmaceutical composition.

In some embodiments, the pharmaceutical composition comprises the amphiphilic emulsifier in an amount of about 6-50% (w/v) of the pharmaceutical composition. In certain embodiments, the pharmaceutical composition comprises the emulsifier in an amount of about 6-10%, about 6-15%, about 6-20, about 6-30%, about 6-40%, about 6-50%, about 10-15%, about 10-20%, about 10-30%, about 10-40%, about 10-50%, about 15-20%, about 15-30%, about 15-40%, about 15-50%, about 20-30%, about 20-40%, about 20-50%, about 30-40%, about 30-50% or about 40-50%, (w/v) of the pharmaceutical composition.

In certain embodiments, the pharmaceutical composition comprises the emulsifier in an amount of about 6%, about 8%, about 10%, about 12%, about 14%, about 16%, about 18%, about 20%, about 25%, about 30%, about 35%, about 40%, about 45% or about 50% (w/v) of the pharmaceutical composition.

b. Lipid Component

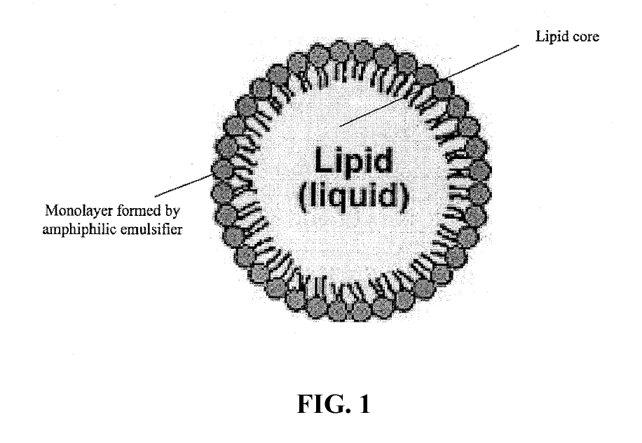

The pharmaceutical composition can comprise a lipid component. As shown in FIG. 1, the lipid component is carried in the hydrophobic core of a SAMS. Examples of lipids include but are not limited to, long-chain and medium chain triglycerides, fatty acyls, glycerolipids, phospholipids, sphingolipids, sterol lipids, prenol lipids, saccharolipids, polyketides, non-natural lipid(s), cationic lipid(s), amphipathic alkyl amino acid derivative, adialkyldimethylammonium, polyglycerol alkyl ethers, polyoxyethylene alkyl ethers, tri-n-octylamine, boric acid, tris(3,5-dimethyl-4-heptyl) ester, triglycerides, diglycerides and other acylglycerols, such as tetraglycerol, pentaglycerol, hexaglycerol, heptaglycerol, octoglycerol, nonaglyceol and decaglycerol, and mixtures thereof. In certain embodiments, the lipid component comprises soybean oil, chia bean oil or algae oil.

In some embodiments, the lipid component is soybean oil. In some embodiments, the lipophilic component is chia bean oil. In some embodiments, the lipophilic component is algae oil. In some embodiments, the lipid component comprises one or more oils rich in omega 3 fatty acids. Oils rich in omega 3 fatty acids include, but are not limited to, chia oil, algae oil, pumpkin oil, flaxseed oil and fish oil. In any given formulation, there could be a mixture of SAMS sizes and emulsifier types and mixture of oils carried in the SAMS.

In some embodiments, the pharmaceutical composition comprises the lipid component in an amount of about 5-70% (w/v) of the pharmaceutical composition. In certain embodiments, the pharmaceutical composition comprises the lipid component in an amount of about 5-70%, 10-70%, 15-70%, 20-70%, 25-70%, 30-70%, 40-70%, 50-70%, 60-70%, 5-60%, 10-60%, 15-60%, 20-60%, 25-60%, 30-60%, 40-60%, 50-60%, 5-50%, 10-50%, 15-50%, 20-50%, 25-50%, 30-50%, 40-50%, 5-40%, 10-40%, 15-40%, 20-40%, 25-40%, 30-40%, 35-40%, 5-35%, 10-35%, 15-35%, 20-35%, 25-35%, 30-35%, 5-30%, 10-30%, 15-30%, 20-30%, 25-30%, 5-25%, 10-25%, 15-25%, 20-25% or 5-20%, 10-20%, 15-20%, 5-15%, 10-15% or 5-10% (w/v) of the pharmaceutical composition. In certain embodiments, the lipid component comprises soybean oil, chia bean oil, algea oil, or combinations thereof, and is included in the pharmaceutical composition in an amount of about 20-30% (w/v) of the pharmaceutical composition. In certain embodiments, the lipid component comprises soybean oil, chia bean oil, algae oil, or combinations thereof, and is included in the pharmaceutical composition in an amount of about 10%, 15%, 20%, 25%, 30%, 35%, 40%, 45%, 50%, 55%, 60%, 65% or 70% (w/v) of the pharmaceutical composition.

c. Carbohydrates Component

The pharmaceutical composition can comprise a carbohydrate or a mixture of carbohydrates. In some embodiments, the pharmaceutical composition comprises a carbohydrate component in the amount of about 1-25% (w/v) of the pharmaceutical composition. In some embodiments, the pharmaceutical composition comprises a carbohydrate component in the amount of about 1, 2, 5, 10, 15, 20 or 25% (w/v) of the pharmaceutical composition. Suitable carbohydrates include, but are not limited to, simple hexose (e.g., glucose, fructose and galactose), mannitol, sorbitol or others known within the art. In some embodiments, the carbohydrate component comprises glucose. In some embodiments, the carbohydrate component consists of glucose.

In some embodiments, the pharmaceutical composition comprises physiological levels of a hexose. Physiological levels of hexose include a hexose concentration of between 0 mM to 60 mM. In some embodiments, the pharmaceutical composition comprises 5 mM glucose. At times, it is desirable to increase the concentration of hexose in order to provide nutrition to cells. Thus, the range of hexose may be expanded up to about 10, 25, 50, 100, 200, 300, 400 or 500 mM within the pharmaceutical composition, if necessary, to provide minimal calories for nutrition.

Other suitable carbohydrates include various saccharides used for medicinal purposes. Examples of these saccharides include, but are not limited to, xylitol, dextrin, glycerin, sucrose, trehalose, glycerol, maltose, lactose, and erythritol.

d. Amino Acid Component

The pharmaceutical composition can comprise an amino acid component. In some embodiments, the pharmaceutical composition comprises an amino acid component at a concentration of 1 nM to 100 mM. In some embodiments, the amino acid component comprises nine essential amino acids (i.e., histidine, isoleucine, leucine, lysine, methionine, phenylalanine, threonine, tryptophan, valine, and combinations thereof). In some embodiments, the amino acid component further comprises alanine, arginine, glycine, proline, serine, tyrosine and combinations thereof. In some embodiments, the amino acid component further comprises glutamate and aspartate. In some embodiments, the amino acid component further comprises one or more amino acids selected from the group consisting of cysteine, acetyl-cysteine, cysteine HCL, taurine, glycyl-glutamine, glycyl-tyrosine, alanyl-glutamine, and combinations thereof.

In some embodiments, the pharmaceutical composition comprises an amino acid component in the amount of about 1%, 2%, 3%, 4%, 5%, 6%, 7%, 8%, 9% or 10% (w/v) of the pharmaceutical composition. Some amino acids, such as histidine, may also be used as a buffering agent.

e. Buffering Agent

The pharmaceutical composition can comprise a buffering agent. The pharmaceutical composition can comprise a biological buffer to maintain the pH of the fluid at the physiological range of pH 7-8. Examples of biological buffers include, but are not limited to, phosphates, histidine, histidine-containing oligopeptides, N-2-Hydroxyethylpiperazine-N′-2-hydroxypropanesulfonic acid (HEPES), 3-(N-Morpholino)propanesulfonic acid (MOPS), 2-([2-Hydroxy-1,1-bis(hydroxymethyl)ethyl]amino)glyci ethanesulfonic acid (TES), 3-[N-tris(Hydroxy-methyl)methylamino]-2-hydroxyethyl]-1-piperazinep ropanesulfonic acid (EPPS), Tris [hydrolymethyl]-aminoethane (THAM), and Tris [Hydroxylmethyl]methyl aminomethane (TRIS).

In some embodiments, the buffering agent is histidine, substituted histidine such as glycylhistidine, imidazole, imidazole derivatives retaining the amphoteric site of the imidazole ring, glycylglycine, carnosine, histidine-containing oligopeptides such as glycylhistidine, his-gly-gly, his-gly-gly-his, gly-his-gly, or mixtures thereof. In some embodiments, the pharmaceutical composition comprises the buffering agent at a concentration range of about 0.001 mM to about 300 mM. In some embodiments, the pharmaceutical composition comprises the buffering agent at a concentration range of about 0.01-100 mM, about 0.01-30 mM, about 0.01-10 mM, about 0.01-3M, about 0.01-1 mM, about 0.01-0.3 mM, 0.01-0.1 mM, 0.01-0.03 mM, 0.1-100 mM, about 0.1-30 mM, about 0.1-10 mM, about 0.1-3M, about 0.1-1 mM, about 0.1-0.3 mM, about 0.3-300 mM, about 0.3-100 mM, about 0.3-30 mM, about 0.3-10 mM, about 0.3-3 mM, about 0.3-1 mM, about 1-300 mM, about 1-100 mM, about 1-30 mM, about 1-10 mM, about 1-3 mM, about 3-300 mM, about 3-100 mM, about 3-30 mM, about 3-10 mM, about 10-300 mM, about 10-100 mM, about 10-30 mM, about 30-300 mM, about 30-100 mM or about 100-300 mM. In some embodiments, the pharmaceutical composition comprises the buffering agent at a concentration range of about 5-20 mM. In some embodiments, the pharmaceutical composition comprises the buffering agent at a concentration range of about 1-10 mM. In some embodiments, the pharmaceutical composition comprises the buffering agent at a concentration of about 10 mM.

In some embodiments, the pharmaceutical composition further comprises L-histidine at a concentration of 1 nM-100 mM. In some embodiments, the pharmaceutical composition comprises L-histidine at a concentration of 0.1-20 mM. In some embodiments, the pharmaceutical composition comprises L-histidine at a concentration of about 0.1 mM. In some embodiments, the pharmaceutical composition comprises L-histidine at a concentration of about 1 mM. In some embodiments, the pharmaceutical composition comprises L-histidine at a concentration of about 10 mM. In some embodiments, the pharmaceutical composition comprises L-histidine at a concentration of about 100 mM.

In some embodiments, the buffering agent is selected from the group consisting of histidine, imidazole, glycylglycine, carnosine, and histidine-containing peptides such as glycylhistidine, gly-his-gly, his-gly-gly and his-gly-gly-his. In some embodiments, the histidine-containing peptides have a histidine content of 20% or higher, 30% or higher, 40% or higher, 50% or higher, 60% or higher, or 70% or higher.

In some embodiments, a buffering agent, such as histidine, glycylglycine or a histidine-containing peptide, is used at a concentration of about 0.5-20 mM of the pharmaceutical composition. In some embodiments, a buffering agent, such as histidine, glycylglycine, histidine-containing peptide, is used at a concentration of about 1-10 mM of the pharmaceutical composition. In some embodiments, a buffering agent, such as histidine, glycylglycine, or histine-containing peptide, is used at a concentration of about 1, 2, 3, 4, 5, 6, 7, 8, 9 or 10 mM of the pharmaceutical composition.

In yet other embodiments, the pharmaceutical composition comprises two or more buffering agents, wherein each is used within the concentration range of 1 uM to 300 mM, 10 uM to 300 mM, 100 uM to 300 mM, 1 mM to 300 mM, 10 mM to 300 mM, 30 mM to 300 mM, 100 mM to 300 mM, 1 uM to 100 mM, 10 uM to 100 mM, 100 uM to 100 mM, 1 mM to 100 mM, 10 mM to 100 mM, 30 mM to 100 mM, 1 uM to 30 mM, 10 uM to 30 mM, 100 uM to 30 mM, 1 mM to 30 mM, 10 mM to 30 mM, 1 uM to 10 mM, 10 uM to 10 mM, 100 uM to 10 mM, 1 mM to 10 mM, 3 mM to 10 mM, 1 uM to 3 mM, 10 uM to 3 mM, 100 uM to 3 mM, 1 mM to 3 mM, 1 uM to 1 mM, 10 uM to 1 mM, 100 uM to 1 mM, 300 uM to 1 mM, 1 uM to 300 uM, 10 uM to 300 uM, 1 uM to 100 uM, 10 uM to 100 uM, 10 uM to 30 uM or 100 uM to 300 uM. In some embodiments, the pharmaceutical composition comprises the two or more buffering agents at a concentration range of about 1 mM to about 10 mM, about 10 mM to about 30 mM or about 30 mM to about 100 mM. In some embodiments, the pharmaceutical composition comprises histidine in the range of 1-10 mM and carnosine in the range of 1-10 mM.

In some embodiments, the pharmaceutical composition uses normal biological components to maintain in vivo biological pH. Briefly, some biological compounds, such as lactate, are capable of being metabolized in vivo and act with other biological components to maintain a biologically appropriate pH in a human or animal. The biological components are effective in maintaining a biologically appropriate pH even at hypothermic temperatures and at essentially bloodless conditions. Examples of the normal biological components include, but are not limited to, carboxylic acids, salts and ester thereof. Carboxylic acids have the general structural formula of RCOOX, where R is an alkyl, alkenyl, or aryl, branched or straight chained, containing 1 to 30 carbons, wherein carbons may be substituted, and X is hydrogen or sodium or other biologically compatible ion substituents that can attach at the oxygen position, or is a short straight or branched chain alkyl containing 1-4 carbons, e.g., —CH₃, —CH₂ CH₃. Examples of carboxylic acids and carboxylic acid salts include, but are not limited to, lactate and sodium lactate, citrate and sodium citrate, gluconate and sodium gluconate, pyruvate and sodium pyruvate, succinate and sodium succinate, and acetate and sodium acetate. Amino acids at concentrations of 100 mM or more can also act as oncotic agents.

f. Electrolyte

The pharmaceutical composition can comprise an electrolyte. In some embodiments, the pharmaceutical composition includes one or more electrolytes. The electrolyte to be used in the present disclosure typically includes various electrolytes used for medicinal purposes. Examples of the electrolyte include, but are not limited to, sodium salts (e.g., sodium chloride, sodium hydrogen carbonate, sodium citrate, sodium lactate, sodium sulfate, sodium dihydrogen phosphate, disodium hydrogen phosphate, sodium acetate, sodium glycerophosphate, sodium carbonate, an amino acid sodium salt, sodium propionate, sodium β-hydroxybutyrate, and sodium gluconate), potassium salts (e.g., potassium chloride, potassium acetate, potassium gluconate, potassium hydrogen carbonate, potassium glycerophosphate, potassium sulfate, potassium lactate, potassium iodide, potassium dihydrogen phosphate, dipotassium hydrogen phosphate, potassium citrate, an amino acid potassium salt, potassium propionate, and potassium β-hydroxybutyrate), calcium salts (e.g., calcium chloride, calcium gluconate, calcium lactate, calcium glycerophosphate, calcium pantothenate, and calcium acetate), magnesium salts (e.g., magnesium chloride, magnesium sulfate, magnesium glycerophosphate, magnesium acetate, magnesium lactate, and an amino acid magnesium salt), ammonium salts (e.g., ammonium chloride), zinc salts (e.g., zinc sulfate, zinc chloride, zinc gluconate, zinc lactate, and zinc acetate), iron salts (e.g., iron sulfate, iron chloride, and iron gluconate), copper salts (e.g., copper sulfate), and manganese salts (for example, manganese sulfate). Among those, particularly preferable are sodium chloride, potassium chloride, magnesium chloride, disodium hydrogen phosphate, dipotassium hydrogen phosphate, potassium dihydrogen phosphate, sodium lactate, sodium acetate, sodium citrate, potassium acetate, potassium glycerophosphate, calcium gluconate, calcium chloride, magnesium sulfate, and zinc sulfate.

In some embodiments, the pharmaceutical composition comprises an electrolyte selected from the group consisting of sodium salts, potassium salts, calcium salts, phosphorus salts and magnesium salts.

Concentration of sodium, potassium, calcium, phosphorus and magnesium ions are typically within the range of normal physiological concentrations of said ions in plasma. In general, the desired concentration of these ions is obtained from the dissolved chloride salts of calcium, sodium and magnesium. The sodium ions may also come from a dissolved organic salt of sodium that is also in solution.

In some embodiments, the pharmaceutical composition comprises a sodium ion concentration in a range from 0 mM to about 600 mM. Hyperosmolarity caused by high concentrations of sodium or other osmolar agents may be useful in victims of head trauma or other causes of increased intracranial pressure. In some embodiments, the pharmaceutical composition comprises a sodium ion concentration in a range of about 90 to 130 mM. In some embodiments, the pharmaceutical composition comprises a sodium ion concentration in an amount of about 102 mM.

In some embodiments, the pharmaceutical composition comprises a calcium concentration in a range of about 0.5 mM to 4.0 mM. In some embodiments, the pharmaceutical composition comprises a calcium concentration in a range of about 2.0 mM to 2.5 mM. In some embodiments, the pharmaceutical composition of the present disclosure does not contain calcium ions.

In some embodiments, the pharmaceutical composition comprises a magnesium ion concentration at range of 0 to 10 mM. In some embodiments, the pharmaceutical composition comprises magnesium ion concentration in a range of about 0.3 mM to 0.45 mM. It is best not to include excessive amounts of magnesium ion in the pharmaceutical composition because high magnesium ion concentrations can negatively affect the strength of cardiac contractile activity. In some embodiments, the pharmaceutical composition comprises subphysiological amounts of magnesium ions. In some embodiments, the pharmaceutical composition does not contain magnesium ion.

In some embodiments, the pharmaceutical composition comprises potassium ions at a concentration of about 4 mM.

In some embodiments, the pharmaceutical composition comprises a chloride ion concentration at a range of 70 mM to 160 mM. In some embodiments, the pharmaceutical composition comprises a chloride ion concentration in a range of 110 mM to 125 mM.

Other sources of ions include, but are not limited to, sodium salts (e.g., sodium hydrogen carbonate, sodium citrate, sodium lactate, sodium sulfate, sodium dihydrogen phosphate, disodium hydrogen phosphate, sodium acetate, sodium glycerophosphate, sodium carbonate, an amino acid sodium salt, sodium propionate, sodium β-hydroxybutyrate, and sodium gluconate), potassium salts (e.g., potassium acetate, potassium gluconate, potassium hydrogen carbonate, potassium glycerophosphate, potassium sulfate, potassium lactate, potassium iodide, potassium dihydrogen phosphate, dipotassium hydrogen phosphate, potassium citrate, an amino acid potassium salt, potassium propionate, and potassium β-hydroxybutyrate), calcium salts (e.g., calcium gluconate, calcium lactate, calcium glycerophosphate, calcium pantothenate, and calcium acetate), magnesium salts (e.g., magnesium sulfate, magnesium glycerophosphate, magnesium acetate, magnesium lactate, and an amino acid magnesium salt), ammonium salts, zinc salts (e.g., zinc sulfate, zinc chloride, zinc gluconate, zinc lactate, and zinc acetate), iron salts (e.g., iron sulfate, iron chloride, and iron gluconate), copper salts (e.g., copper sulfate), and manganese salts (for example, manganese sulfate). Among those, particularly preferable are sodium chloride, potassium chloride, magnesium chloride, disodium hydrogen phosphate, dipotassium hydrogen phosphate, potassium dihydrogen phosphate, sodium lactate, sodium acetate, sodium citrate, potassium acetate, potassium glycerophosphate, calcium gluconate, calcium chloride, magnesium sulfate, choline chloride and zinc sulfate.

g. Vitamin

The pharmaceutical composition can comprise a vitamin. In some embodiments, the pharmaceutical composition comprises one or more vitamins selected from the group consisting of vitamin A, vitamin D, vitamin E, vitamin C, vitamin B1 (thiamine), vitamin B2 (riboflavin), vitamin B6 (pyridoxine), vitamin B12 (cyanocobalamin), vitamin K, folic acid, niacin, biotin and panothenic acid. In some embodiments, the pharmaceutical composition comprises one or more trace elements selected from the group consisting of manganese, chromium, selenium, copper and zinc. Additional vitamins and trace minerals may be added to the pharmaceutical composition in conditions of possible deficiencies or need. For example, additional vitamin C and zinc may be added to promote wound healing. Additional zinc may also be incorporated into the pharmaceutical composition for individuals with high volume stool or fistula losses. Additional vitamin B12 can also be added to the pharmaceutical composition if the individual presents with needs that are not addressed by the standard vitamin preparation.

h. Other Therapeutic Agents

The pharmaceutical composition can comprise other therapeutic agents. In some embodiments, the pharmaceutical composition comprises one or more other therapeutic agents that are compatible with TPN/TPA. Examples of such therapeutic agents include, but are not limited to, anti-inflammatory agents, such as histidine, albumin, (+) naloxone, prostaglandin D₂, molecules of the phenylalkylamine class, interferon; interferon derivatives comprising betaseron, β-interferon; prostane derivatives comprising iloprost, cicaprost and glucocorticoids; immunomodulatory agents such as cyclosporine A, methoxsalene, sulfasalazine, azathioprine, methotrexate; lipoxygenase inhibitors comprising zileutone, MK-886, WY-50295, SC-45662, SC-41661A, BI-L-357; leukotriene antagonists; peptide derivatives comprising ACTH and analogs thereof; soluble TNF-receptors; anti-TNF-antibodies; soluble receptors of interleukins or other cytokines; antibodies against receptors of interleukins or other cytokines, T-cell-proteins, and calcipotriols and analogues thereof; antibiotics such as penicillin, cloxacillin, dicloxacillin, cephalosporin, erythromycin, amoxicillin-clavulanate, ampicillin, tetracycline, trimethoprim-sulfamethoxazole, chloramphenicol, ciprofloxacin, aminoglycoside (e.g., tobramycin and gentamicin), streptomycin, kanamycin and neomycin; sulfa drugs; land monobactams; anti-viral agents, such as amantadine hydrochloride, rimantadin, acyclovir, famciclovir, foscarnet, ganciclovir sodium, idoxuridine, ribavirin, sorivudine, trifluridine, valacyclovir, valgancyclovir, pencyclovir, vidarabin, didanosine, stavudine, zalcitabine, zidovudine, interferon alpha, and edoxudine; anti-fungal agents such as terbinafine hydrochloride, nystatin, amphotericin B, griseofulvin, ketoconazole, miconazole nitrate, flucytosine, fluconazole, itraconazole, clotrimazole, benzoic acid, salicylic acid, voriconazole, caspofungin, and selenium sulfide; vessel expanders such as alcohols and polyalcohols; mediators of vascular potency and immunomoduators, such as prostaglandins, leukotrienes, pro-opiomelanocortin fragments and platelet activating factors; and other therapeutic agents such as ranitidine (zantac), famotidine (pepcid) and insulin.

In some other embodiments, the pharmaceutical composition comprises a potassium channel blocker, which is capable of inhibiting programmed cell death by preventing potassium efflux.

In certain embodiments, the pharmaceutical composition comprises anti-cancer drugs and/or intracellular signal molecules, such as cAMP and diacylglycerol. In some embodiments, the pharmaceutical composition comprises proopiomelanocortin fragments, such as beta endorphin, melonocyte stimulating hormone enkephalins or opiates to modify the immune response and to provide analgesia. Beta endorphin may also be used at a concentration of 0.01-120 nm in the pharmaceutical composition. In some embodiments, the pharmaceutical composition comprises beta endorphin in the concentration range of 0.1-10 nm. In some embodiments, the pharmaceutical composition comprises beta endorphin at a concentration of about 0.2, 0.5, 1, 2 or 5 nm.

In some embodiments, the pharmaceutical composition comprises one or more neurotrophic agents for treatment of psychiatric disease or prevention of psychiatric disease. Neurotrophic agents are important for survival, growth, or differentiation of discrete neuronal populations. Examples of neurotrophic agents include, but are not limited to, factors in the neurotrophin family, such nerve growth factor (NGF), brain-derived neurotrophic factor (BDNF), neurotrohin-3 (NT-3) and neurotrohin-4 (NT-4); factors in the CNTF family, such as ciliary neurotrophic factor (CNTF), leukemia inhibitory factor (LIF) and interleukin-6 (IL-6); and factors in the GDNF family, such as glial cell line derived neurotrophic factor (GDNF), neurturin (NTN), artemin (ART) and persephin (PSP).

i. Antioxidant

The pharmaceutical composition can comprise an antioxidant. In certain embodiments, the pharmaceutical composition may comprise one or more antioxidants. Examples of antioxidants include, but are not limited to, sodium hydrogen sulfite, sodium sulfite, sodium pyrosulfite (e.g., sodium metabisulfite), rongalite (CH2OHSO2Na), ascorbic acid, sodium ascorbate, erythorbic acid, sodium erythorbate, cysteine, cysteine hydrochloride, homocysteine, glutathione, thioglycerol, α-thioglycerin, sodium edetate, citric acid, isopropyl citrate, potassium dichloroisocyanurate, sodium thioglycolate, sodium pyrosulfite 1,3-butylene glycol, disodium calcium ethylenediaminetetraacetate, disodium ethylenediaminetetraacetate, an amino acid sulfite (e.g, L-lysine sulfite), butylhydroxyanisole (BHA), butylhydroxytoluene (BHT), propyl gallate, ascorbyl palmitate, vitamin E and derivatives thereof (e.g., dl-α-tocopherol, tocopherol acetate, natural vitamin E, d-δ-tocopherol, mixed tocopherol, and trolox), guaiac, nordihydroguaiaretic acid (NDGA), L-ascorbate stearate esters, soybean lecithin, palmitic acid ascorbic acid, benzotriazol, and pentaerythrityl-tetrakis[3-(3,5-di-t-butyl-4-hydroxyphenyl)propionate]2-mercaptobenzimidazole. Among those, preferable are sodium hydrogen sulfite, sodium sulfite, ascorbic acid, homocysteine, dl-α-tocopherol, tocopherol acetate, glutathione, and trolox.

j. Other Components

The pharmaceutical composition can comprise other components. In addition to the components discussed above, the pharmaceutical composition may further comprise other additives that include, but are not limited to, lactate, glycerol and mannitol. In certain embodiments, the pharmaceutical composition may comprise beneficial anions such as lactate or glutamate. Hypertonic lactate containing compositions have been found to be effective in reducing brain edema in patients with acute hemodynamic distress. In some embodiments, the pharmaceutical composition comprises 250 to 2400 mM of lactic acid or lactate. In some embodiments, the pharmaceutical composition comprises 250 to 500 mM, 500 to 1000 mM, 1000 to 1500 mM, 1500 to 2000 mM or 2000 to 2400 mM of lactic acid or lactate. In some embodiments, the pharmaceutical composition comprises 0-30% (w/v) glycerol and/or 0-30% (w/v) mannitol.

In certain embodiments, the pharmaceutical composition may comprise substituted cations. For example, the pharmaceutical composition may comprise choline to substitute sodium ions.

In some embodiments, the pharmaceutical composition comprises, in a one liter volume, 5-70% (w/v) soybean oil, chia bean oil, algae oil, pumpkin oil, or combinations thereof; 6% (w/v) or more egg phospholipids, soybean lecithin or a combination thereof; 0-100 gram glucose; Na⁺ at 20-155 mEq, K⁺ at 0-80 mEq, Ca⁺⁺ at 0-360 mEq, Mg⁺⁺ at 0-240 mE, Acetate⁻ at 0-230 mEq, Cl⁻ 0-155 mEq, P-0-465 mg, MVI-12 1.4 ml, 200 μg folic acid, 5 μg vitamin B12 and 70 mg vitamin C. In some embodiments, trace elements are supplemented as needed.

In some embodiments, the pharmaceutical composition comprises 20-30% (w/v) soybean oil, chia bean oil or algae oil, 6-12% (w/v) egg phospholipid and 1-5% (w/v) amino acids. In some embodiments, the above pharmaceutical composition further comprises a buffering agent at a concentration of 1-100 mM. In some embodiments, the buffering agent comprises 1-10 mM histidine, 1-10 mM glycylglycine, 1-10 mM carnosine, 1-10 mM glycylhistidine, 1-10 mM gly-his-gly, 1-10 mM other polyhistidine, polyglycine or his/gly-containing peptides of 3-10 amino acids or combinations thereof. In some embodiments, the above pharmaceutical composition further comprises lysine at a concentration of 1-10 mM. In some embodiments, the above pharmaceutical composition further comprises lysine at a concentration of about 4 mM. In some embodiments, the pharmaceutical composition further comprises glucose in an amount in the range of 1-10% (w/mw) of the total composition.

The pharmaceutical composition can be free of Al⁺⁺⁺, as well as hemoglobin, derivatives of hemoglobin, perfluorocarbon and derivatives of perfluorocarbon. Because Al⁺⁺⁺ is toxic to bone, brain, hematopoieisis, heme synthesis, globulin synthesis, iron absorption and metabolism, and fetal development, all oils and other components must have the minimum amount of Al⁺⁺⁺ possible. In certain embodiments, the pharmaceutical composition contains Al⁺⁺⁺ at a concentration of less than 25 μg/l, 20 μg/l, 10 μg/l or 5 μg/l. In other embodiments, the pharmaceutical composition is free of Al⁺⁺⁺, i.e., undetectable by conventional methods. In certain embodiments, Ca⁺⁺ and/or Mg⁺⁺ are added to the pharmaceutical composition just prior to use (i.e., within 24 hours prior to use).

k. Self-Assembling Amphiphilic Molecular Structure (SAMS)

The pharmaceutical compositions can comprise SAMS. FIG. 1 shows an embodiment wherein the lipid component and the amphiphilic emulsifier form lipid-containing micelles (LM), lipid-containing liposomes (LL) or combinations thereof that are surrounded by the water (shown as the white space around the amphiphilic molecules). The amphiphilic emulsifier molecules occupy the periphery of the lipid boundary. The lipophilic ends of the amphiphilic emulsifier molecules are directed inward toward the lipid and the polar ends of the amphiphilic emulsifier molecules are directed outward toward the polar carrier fluid. Because hydrophobic gases, such as oxygen, preferentially dissolve in the lipid core of the micelles and/or liposomes relative to water or other aqueous environments, the pharmaceutical compositions of the present disclosure provides the ability to form a stable suspension of SAMS so that the composition may be stored at room temperature or under refrigeration for an extended period of time. In some embodiments, the compositions of the present disclosure are stable at room temperature for 10, 20, 30, 60, 90 days or longer. In some embodiments, the composition of the present disclosure is stable under refrigeration (typically 4-8° C.) for 1, 3, 6, 9 or 12 months.

In some embodiments, the pharmaceutical composition comprises SAMS having diameters of 120 nm or less. In some embodiments, the pharmaceutical composition comprises SAMS having diameters of 80 nm or less, 60 nm or less, 40 nm or less, 30 nm or less, or 20 nm or less. In some embodiments, the pharmaceutical composition comprises SAMS having diameters in the range of 1-120 nm, 1-100 nm, 1-80 nm, 1-60 nm, 1-40 nm, 1-30 nm, 1-20 nm, 20-120 nm, 20-100 nm, 20-80 nm, 20-60 nm, 20-40 nm, 20-30 nm, 30-120 nm, 30-100 nm, 30-80 nm, 30-60 nm, 30-40 nm, 40-120 nm, 40-100 nm, 40-80 nm, 40-60 nm, 60-120 nm, 60-100 nm, 60-80 nm, 80-100 nm or 100-120 nm. The use of SAMS with a diameter of 120 nm or less allows the passage of the SAMS through the sinusoids of the liver much more readily than larger SAMS. This can eliminate the congestion seen with SAMS. Further, because the SAMS of the present disclosure pass more readily through the liver sinusoids, there is no reason to limit the percentage of the total body caloric intake that comes from them. This eliminates the need for high glucose. Furthermore, without the need for a high glucose, the TPN/TPA SAMS of the present disclosure can be administered via a peripheral vein. This can greatly reduce the difficulty, discomfort and danger of giving TPN/TPA.

The use of SAMS with a diameter of 120 nm or less allows the passage of the SAMS through the reticulendothelial system much more readily than larger SAMS. This can eliminate the splenic congestion seen with larger SAMS currently used for TPNs/TPAs. In some embodiments, the pharmaceutical composition comprises a lipid component, which consists of an oil or a mixture of oils with high levels of docosahexaenoic acid and eicosapentaenoic acid (such as algae oil or chia bean oil). Using these types oils can reduce the inflammatory response of the Kupffer cells in the liver.

The SAMS of the present disclosure can be free-moving SAMS that are not encapsulated in any type of particles. Further, the wall of the SAMS can comprise a single layer, a double layer, a multi-layer, or combinations thereof, of the amphiphilic emulsifier molecules. Thus, the SAMS may easily merge with the cell membrane of a tissue that the SAMS comes into contact with. Further, the SAMS within the pharmaceutical composition can be free of hemoglobin, derivatives of hemoglobin, perfluorocarbon and derivatives of perfluorocarbon.

### 3. Preparation of the Pharmaceutical Composition

The pharmaceutical composition may be prepared by mixing the amphiphilic emulsifier, the water carrier, and the lipid component and any other components, to form an emulsion. Commonly used mixing methods include, but are not limited to, stirring, shaking, homogenization, vibration, microfluidization and sonication. In some embodiments, the present disclosure provides a method for preparing the pharmaceutical compositions. The method comprises the steps of combining water, a lipid component and an emulsifier component to form a liquid mixture having 30-80% water, 5-70% (w/v) lipid component and 6-20% (w/v) emulsifier component; homogenizing the liquid mixture under conditions that produce micelles and/or liposomes in the size range of 0.1-120 nm.

In some embodiments, the pharmaceutical composition is prepared according to the following recipe:

For 1 liter of premixed TPN/TPA, prepare a mixture containing the following components:

Oil - soybean, chia, algae, pumpkin, in various combinations 10-60% w/v
Emulsifier - egg phospholipids (egg lecithins), soybean lecithin other
amphiphillic molecule at 6% w/v or more

| Glucose | 0-100 | grams |
|---|---|---|
| Sodium | 20-155 | mEq |
| Potassium | 0-5 | mEq |
| Calcium | 360 | mg |
| Magnesium | 240 | mg |
| Acetate | 0-230 | mEq |
| Chloride | 0-155 | mEq |
| Sodium Phosphate | 0-465 | mg |
| Retinol | 3330 IU (1 IU = 0.3 μg retinol) | |
| Calciferol | 200 IU (40 IU = 1 μg calciferol) | |
| Tocopherol | 10 IU (1 IU = 1 mg D,L-α-tocopherylacetate = 0.67 | |
| | mg D-α-tocopherol) | |
| Phylloquinone | 2-4 | mg |
| Ascorbic acid | 100 | mg |
| Folic acid | 400 | μg |
| Niacin | 40 | mg |
| Riboflavin | 3.6 | mg |
| Thiamine | 3.0 | mg |
| Pyridoxine | 4.0 | mg |
| Cyanocobalamin | 5.0 | μg |
| Pantothenic acid | 15 | mg |
| Biotin | 60 | μg |
| vitamin B12 | 5 | μg |
| vitamin C | 70 | mg |
| Iron | 1-2 | mg |
| Zinc | 2.5-4 | mg |
| Copper | 0.5-1.5 | mg |
| Chromium | 10-15 | μg |
| Manganese | 0.15-0.8 | mg |
| Molybdenum | 50-400 | μg |
| Selenium | 20-50 | μg |
| Iodide | 0.15 | mg |
| Fluoride | 1 | mg |
| Amino acids | 75-128 | grams |

Add water as needed to make final volume of 1 liter.
The mixture was homogenized under conditions that produce micelles and/or liposomes with diameters of 120 nm or less.

### 4. Kits

Another aspect of the present disclosure relates to a TPN/TPA kit. In one embodiment, the TPN/TPA kit comprises a pharmaceutical composition of the present disclosure and an intravenous infusion (IV) set.

Another aspect of the present disclosure relates to a kit for treatment of a human or animal subject to raise its blood pressure. In some embodiments, the kit is for the treatment of a human or animal subject intended to raise its blood pressure. The kit can comprise a two component plastic bag, a pharmaceutical composition of the present disclosure and an oxygenation device. The pharmaceutical composition may be placed in the larger component of the two component plastic bag and the plasma in powder form placed in the smaller component of the two component plastic bag. Attached to the kit can be an oxygenation device.

### 5. Methods of Treatment

The pharmaceutical compositions of the present disclosure are useful for treating and preventing certain diseases and disorders in humans and animals. For example, the pharmaceutical compositions of the present disclosure, including but not limited to those specified in the examples, can be used to treat hemodynamic stability, such as intravascular volume loss, excessive nitric oxide production, and/or blood pressure in a subject.

Hemodynamic stability has been achieved when tissue perfusion is sufficient to provide adequate oxygen delivery to tissues to sustain aerobic metabolism. Oxygen delivery is a function of the oxygen content of the blood, as well as the intravascular volume, cardiac contractile force and vascular tone. When there is blood loss or loss of fluid from the intravascular space due to diarrhea, vomiting or other means of causing actual or relative hypovolemia, for example, after hemorrhagic shock or conditions related to lack of blood volume supply, there is a volume deficit that reduces tissue perfusion. In addition, there is an increase in the capillary permeability largely due to the formation of pores that have a diameter of 30 nm. This capillary leak exacerbates the loss of intravascular volume and reduces oxygen delivery.

Another important consequence of severe loss of intravascular volume through loss of blood or other fluids from the vascular space is the excessive production of nitric oxide. Excessive production of nitric oxide results in a decrease in cardiac contractility, vascular tone and blood pressure (Langenbecks Arch Surg. 386 (4), 302-308, 2001; Zhonghua Shao Shang Za Zhi, 22(5):343-346, 2006, both of which are incorporated by reference in their entirety).

Also, when there is blood loss or loss of fluid from the intravascular space due to diarrhea, vomiting or other means of causing actual or relative hypovolemia, for example, after hemorrhagic shock or conditions related to lack of blood volume supply, there is a volume deficit that reduces tissue perfusion. The pharmaceutical compositions of the present disclosure comprising smaller and more stable SAMS (average micelle diameter of 50-120 nm), NaCl (102 mM), Na (L) lactate (35 mM), L-histidine (50 mM or less) made with egg phospholipid or egg lecithin beyond 2% (w/v) including in an amount of 6% (w/v) or higher is effective in the treatment of conditions brought about as a result of loss of intravascular volume and/or the excessive production of nitric oxide or other mediators to which it binds or that are absorbed by it. Its primary component is a SAMS comprised of phospholipids with purified soybean oil inside the hydrophobic core of the micelle. The SAMS is a colloid that is large enough to remain in the intravascular space and restore the intravascular volume and tissue perfusion. In addition, the SAMS absorbs and readily releases nitric oxide. This reduces the nitric oxide that is available to produce adverse effects on blood vessel reactivity to endogenous or exogenous vaso-constrictors and on cardiac output. Liposomes would have the same properties as the micelles because they also provide volume that is too large to leak from the intravascular space and their lipid component can also absorb and readily release nitric oxide and bind to other mediators.

While lecithin-coated perfluorocarbons (PFCs) are known in the art as relatively stable blood substitutes, PFCs are known to have many limitations and adverse medical consequences (U.S. Pat. No. 4,423,077, which is incorporated by reference in its entirety). At a minimum, hypovolemic shock resuscitation fluids are rapidly infused into patients to raise the blood pressure so that tissue perfusion is improved without doing harm. When perfluorocarbons are rapidly infused they do the opposite. PFCs can decrease the blood pressure and promote hemodynamic instability (J Surg Res. 2015 Apr. 28. pii: S0022-4804(15)00495-3, which is incorporated by reference in its entirety). It is also known in the art that PFCs produce an intense inflammatory reaction (Anesth Analg. 2008 January; 106(1):24-31, which is incorporated by reference in its entirety). It has been shown in the art that trying to offset the hypotensive effect of PFCs cannot be done by mixing them in an emulsion (such as a PFC emulsion) Hextend (J Surg Res. 2015 Apr. 28. pii: S0022-4804(15)00495-3, which is incorporated by reference in its entirety). Others in the art have also shown that encapsulating the PFC in a microcapsule was inadequate and failed to produce optimal results (Results Pharma Sci. 2014 Apr. 30; 4:8-18, which is incorporated by reference in its entirety). Further, it is known within the art that PFCs are eliminated via the lungs and are very effective greenhouse gases. It is likely that for the aforementioned limitations and many other reasons, perfluorocarbon products for the treatment of stroke and traumatic brain injury have abandoned these products.

In addition, the pharmaceutical compositions of the present disclosure can be used for raising the blood pressure in euvolemic or hypovolemic human and animal subjects. Moreover, the lipid emulsions comprise SAMS that are stable in spite of their low zeta potential (i.e., less than 30 mV). For example, the pharmaceutical compositions of the present disclosure comprising smaller and more stable SAMS (average diameter of 1-120 nm), NaCl (102 mM), Na (L) lactate (35 mM), L-histidine (50 mM or less) made with egg phospholipid or egg lecithin beyond 2% (w/v) including in an amount of 6% (w/v) or higher, is effective in increasing the blood pressure in euvolemic, hypovolemic or hypervolemic patients. In some embodiments, the pharmaceutical compositions used for increasing the blood pressure in a subject, may comprise the following components wherein all % is w/v: soybean oil 20%, egg lecithin 12%, NaCl 102 mM, Na (L) lactate 35 mM, (L) Histidine 0.1 mM, water, pH=7-7.5. In some embodiments, treatment of a human or animal subject to increase its blood pressure using the pharmaceutical composition of the present disclosure comprises infusing the pharmaceutical composition intravenously or intra-arterially. The volume of infusion is determined by reaching the goals set by the treating health professional. Examples, of goals are a blood pressure of 90 systolic, the maximum stroke volume index, or a CVP of 9 mm H₂O.

The pharmaceutical compositions of the present disclosure, including but not limited to those specified in the examples, are useful for opening constricted or blocked blood vessels in a subject. For example, the pharmaceutical compositions of the present disclosure comprising smaller and more stable SAMS (average diameter of 90-120 nm), NaCl (102 mM), Na (L) lactate (35 mM), L-histidine (10 mM or less) made with egg phospholipid or egg lecithin beyond 2% (w/v) including in an amount of 6% (w/v) or higher, may be used to open constricted or blocked blood vessels in a human or animal subject.

The pharmaceutical compositions of the present disclosure, including but not limited to those specified in the examples, are useful for providing TPN/TPA to a subject. For example, TPN/TPA is given to subjects who cannot receive nutrition via the gastrointestinal system. This could occur because of conditions such as the presence of high output gastrointestinal fistulas, bowel length that is too low to provide sufficient absorptive surface, severe inflammatory bowel disease, necrotizing bowel disease or prolonged and high volume diarrhea.

TPN or TPA is a liquid mixture of glucose, lipids, amino acids, electrolytes, vitamins, minerals, trace elements and other additives that is given intravenously in order to provide nutrition when the gastrointestinal tract is unavailable. Typically, a mechanical pump under computer control is used to dispense the TPN/TPA fluid over a period of from twelve to sixteen hours a day in a hospital setting. Presently, the use of portable pumps with rechargeable batteries and portable component packs allows administration of TPN/TPA at home and certain mobility for patients during administration periods.

However, there are significant problems with traditional TPN/TPA formulations. First, traditional TPN/TPA formulations require administration via a central vein such as the superior or inferior vena cava. This route of administration is necessary because of the high osmolarity of traditional TPN/TPA formulations. This high osmolarity is due to the presence of high concentrations of glucose, which is necessary in order to provide sufficient calories. It is generally known within the art that there is a limitation in the percentage of the total caloric need that can be given as lipids. If traditional TPN/TPA is given via a peripheral vein as opposed to a central vein, the high osmolarity causes severe injury to the peripheral vein. For example, resultant inflammation due to hyperosmolar damage to the vascular walls can make the vein unusable and a possible source of infection. However in order to place a cannula into the central vein, a health care professional must place it into the neck, chest or groin. Placement of these central lines requires a high level of skill and time. It may also require special instrumentation to guide placement of the needle used to penetrate into the area of the central vein, because the needle could injure surrounding structures, such as major arteries and the lungs. Cannulation of the central venous system may also cause severe, life threatening infection of the blood.

Moreover, current TPN/TPA formulations contain SAMS in the size range of about 300 nm. The SAMS may cause congestion of the spleen. The spleen eliminates bacteria, but if the splenic sinusoids are congested with SAMS, the bacteria are not removed from the circulation by the spleen which, in turn, increases the chance of infection.

Finally, traditional TPN/TPA formulations contain lipid and non-lipid components that are stored separately. The lipid components are mixed with the non-lipid components on the day of use, because of the instability of lipid SAMS in the non-lipid aqueous solution. The pre-infusion mixing procedure is not only inconvenient, but also increases the possibility of contamination, especially in a home administration setting. Accordingly, there exists a need to develop a TPN/TPA formulation that is safer and easier to use.

One aspect of the present disclosure relates to pharmaceutical compositions for TPN or TPA. The composition comprises water as a pharmaceutically acceptable carrier, a lipid component in an amount of 5-70% (w/v) of the pharmaceutical composition, and an amphiphilic emulsifier in an amount of 6% (w/v) or more of the pharmaceutical composition. The lipid component and the amphiphilic emulsifier form free-moving SAMS in the water carrier, wherein the SAMS have diameters of 120 nm or less. The pharmaceutical composition is free of hemoglobin, derivatives of hemoglobin, perfluorocarbon and derivatives of perfluorocarbon.

The TPN/TPA of the present disclosure is based on the novel finding that the use of phospholipids in an amount of 6% (w/v) or higher results in SAMS with diameters of 120 nm or less. Furthermore, these small SAMS are stable in suspensions, regardless of a low zeta potential. Current TPN/TPA products separate the lipid and aqueous phases and mix the two phases in pharmacies within 24 hours of infusion. With this discovery it is possible to premix the two phases and leave it on the shelf for an extended period of time. Premixing can eliminate the need for a multitude of pharmacy resources that go into making TPN/TPA. In addition, premixing reduce the possibility of error in mixing and contamination.

SAMS in the Peripheral Total Parenteral Nutrition or Peripheral Total Parenteral Nutrition are stable when mixed with further components including, but not limited to, electrolytes, glucose and amino acids. Therefore, the TPN/TPA does not have to be mixed the day of use. SAMS in the TPN/TPA are stable, in part, because of the use of high concentration (6% (w/v) or higher) of emulsifer. The TPN/TPA of the present disclosure can give a higher percentage of calories as lipid because the SAMS are smaller. Smaller SAMS corresponds to less uptake and sequestration by the reticuloendothelial system.

Additionally, there is no need for high concentrations of glucose. Accordingly, TPN/TPA can be isotonic and given through a peripheral vein. Currently, hypertonic TPN/TPA must be given via a central vein, and is a very involved procedure with significant risk and cost, which can increase risk of sepsis. This is avoided with peripheral vein administration. In some embodiments, the TPN/TPA of the present disclosure uses algae oil or other oil high in omega 3 fatty acids. The omega 3 fatty acids found in algae oil could reverse inflammation of the liver that may occur in critical illness.

In some embodiments, the method comprises the steps of administering the TPN/TPA composition of the present disclosure to a subject in need thereof an effective amount of the composition of the present disclosure. In some embodiments, the TPN/TPA composition of the present disclosure is administered intravenously through a central venous catheter. In other embodiments, the TPN/TPA composition of the present disclosure is administered through a peripheral vein. The amount and infusion rate of the composition is calculated based on the patient's need. The contents of the TPN/TPA composition may be adjusted to meet the special need of each patient, while maintaining the desired osmolarity and daily caloric requirement of the patient.

The pharmaceutical compositions of the present disclosure, including but not limited to those specified in the examples, are useful for conducting renal dialysis or coronary bypass in a subject. For example, the pharmaceutical compositions of the present disclosure comprising smaller and more stable SAMS (average diameter of 1-120 nm), NaCl (102 mM), Na (L) lactate (35 mM), L-histidine (1 mM or less) made with egg phospholipid or egg lecithin beyond 2% (w/v) including in an amount of 6% (w/v) or higher, can be utilized to prevent dangerous hypotension that may occur in patients while on renal dialysis. In some embodiments, the pharmaceutical compositions of the present disclosure used for renal dialysis may comprise the following components wherein all % is w/v: soybean oil 20%, egg lecithin 12%, NaCl 102 mM, Na (L) lactate 35 mM, (L) Histidine 0.1 mM, water, pH=7-7.5. This fluid should be used as previous fluids were used for dialysis. In hemodialysis it should be passed through a dialyzer membrane. In peritoneal dialysis, it should be infused into the peritoneal cavity via a peritoneal dialysis catheter. See the typical components of a representative renal dialysis fluid in the table immediately below.

TABLE 1

| Composition | Hemodialysis | | Peritoneal dialysis | |
|---|---|---|---|---|
| Na | 136-140 | mEq/L | 132 | mEq/L |
| Cl | 99-110 | mEq/L | 96 | mEq/L |
| K | 0-4 | mEq/L | 0-10 | mEq/L |
| Ca | 2.5 | mEq/L | 3.5 | mEq/L |
| Mg | 0.5-1 | mEq/L | 0.5 | mEq/L |
| Acetate | 2.5-5 | mEq/L | — | |
| Bicarbonate | 27-39 | mEq/L | — | |
| Lactate | — | | 40 | mEq/L |
| Glucose | 200 | mg/dL | 1500-4250 | mg/dL |

The pharmaceutical compositions of the present disclosure, including but not limited to those specified in the examples, are useful for transporting therapeutic gases, such as oxygen, nitric oxide, hydrogen sulfide, xenon, argon, and/or carbon dioxide in a subject. For example, the pharmaceutical compositions of the present disclosure comprising smaller and more stable SAMS (average diameter of 1-120 nm), NaCl (102 mM), Na (L) lactate (35 mM), L-histidine (1 mM or less) made with egg phospholipid or egg lecithin beyond 2% (w/v) including in an amount of 6% (w/v) or higher, can be used to transport therapeutic gases such as oxygen, nitric oxide, hydrogen sulfide, xenon, argon or carbon monoxide. Oxygen can be used to promote aerobic metabolism throughout the body or to specific regions, nitric oxide may be used to dilate blood vessels in peripheral vascular disease, coronary artery disease, ischemic stroke or in sickle cell disease as examples. Hydrogen sulphide may be used to induce a state of suspended animation or to slow the aging process of the whole body or of an organ in vivo or ex vivo. Xenon has anesthetic properties. Carbon monoxide may be used to inhibit apoptosis of the entire body or of an organ in vivo or ex vivo. Each of these gases may be loaded onto the micelles and applied either to the whole body, a region of the body or to an organ in-vivo or ex-vivo.

The pharmaceutical compositions of the present disclosure, including but not limited to those specified in the examples, are useful for conducting an exchange transfusion to replace plasma or whole blood that contains harmful mediators, prions, viruses, bacteria, fungi, chemical or biological agents of warfare, cancer cells or other blood stream born agents in a subject. For example, the pharmaceutical compositions of the present disclosure comprising smaller and more stable SAMS (average diameter of 90-120 nm), NaCl (102 mM), Na (L) lactate (35 mM), L-histidine (1 mM or less) made with egg phospholipid or egg lecithin beyond 2% (w/v) including in an amount of 6% (w/v) or higher, may be used for exchange transfusion. A patient's blood, lymph or cerebrospinal fluid, may be removed and replaced by the pharmaceutical compositions of the present disclosure comprising stabilized SAMS in order to remove toxic molecules or bacteria, viruses, cells or prions or other living pathogens from the bloodstream. Circulating cancer cells could similarly be removed.

The pharmaceutical compositions of the present disclosure, including but not limited to those specified in the examples, are useful for conducting Extracorpeal Membrane Oxygenation (ECMO) in a subject. For example, the pharmaceutical compositions of the present disclosure comprising smaller and more stable SAMS (average diameter of 90-120 nm), NaCl (102 mM), Na (L) lactate (35 mM), L-histidine (1 mM or less) made with egg phospholipid or egg lecithin beyond 2% (w/v) including in an amount of 6% (w/v) or higher, may be used for Extracorporeal Membrane Oxygenation (ECMO). The pharmaceutical compositions of the present disclosure comprising stabilized SAMS may be used to provide a gas carrying circulating fluid to replace blood in patients who are on ECMO for a variety of reasons that include respiratory or cardiac failure.

The pharmaceutical compositions of the present disclosure, including but not limited to those specified in the examples, are useful for treating septic shock and irreversible shock in a subject. Currently there is no treatment for irreversible septic shock. The pharmaceutical compositions of the present disclosure comprising smaller and more stable SAMS (average diameter of 90-120 nm), NaCl (102 mM), Na (L) lactate (35 mM), L-histidine (50 mM or less) made with egg phospholipid or egg lecithin beyond 2% (w/v) including in an amount of 6% (w/v) or higher, could be used to treat septic shock and irreversible shock due to sepsis and or severe blood loss. Sepsis is an abnormal physiological state caused by the body's response to bacterial infection. This response consists of the activation of cells such as neutrophils and macrophages and the release of mediators into the blood stream such as interleukin 1, tumor necrosis factor and nitric oxide. This response includes fever, tachycardia, organ damage, depression of cardiac function and hypotension. Nitric oxide is the primary mediator of hypotension (i.e., decreased blood pressure). The hypotension of irreversible septic shock is a result of dilation of blood vessels and the depression of cardiac output. Excessive nitric oxide is a primary cause of these adverse effects and the absence of response to pressors and inotropes (Pharmacol. 1993 March; 108(3):786-92; Life Sci. 2007 Aug. 16; 81(10):779-93. Epub 2007 Aug. 2, all of which are incorporated by reference in their entirety).

When the blood pressure decreases below a level compatible with survival, it is increased by the infusion of fluid which may be crystalloid, colloid or blood. However, there is a limit to the amount of fluid that can be infused. Too much fluid will create a load against the outflow of blood from the heart resulting in a decrease in blood pressure, tissue perfusion, and pulmonary edema. Often when this limit has been reached, a presser such as Levophed that increases blood pressure by narrowing the walls of the blood vessels or an inotrope such as Dopamine that increases cardiac output is used to increase the blood pressure. When neither fluid nor medications are effective in increasing the blood pressure, a state of irreversible shock exists, and this can to death. The SAMS of the present disclosure have a diameter that is large enough to remain inside the intravascular space rather than leak out, which occurs with the smaller albumin and sodium ions. Therefore, irreversible septic shock could be reversed by the pharmaceutical compositions of the present disclosure due to its ability to act as a low affinity high capacity reservoir for nitric oxide and its ability to provide the necessary volume in the intravascular space needed to provide for effective circulation. In addition, the absorption of nitric oxide in the heart would remove the depressive effect it has on cardiac contraction.

Nitric oxide is a significant mediator of hypotension and cardiac dysfuntion in sepsis or after severe blood loss. This condition may become unresponsive to the administration pressors such as norepinephrine or inotropes such as dobutamine or blood, blood products, fluid resuscitation with crystalloids such as Ringer's lactate or colloids such as albumin. Nitric oxide is readily taken up and released by the SAMS of the present disclosure, for example, the SAMS of the present disclosure can reversibly absorb nitric oxide. (J Chromatogr B Analyt Technol Biomed Life Sci. 2011 Jun. 1; 879(19): 1513-1518, which is incorporated by reference in its entirety). Therefore the pharmaceutical compositions of the present disclsoure comprising SAMS would be able to reduce the effective concentration of nitric oxide and reverse what is now considered an irreversible state.

The pharmaceutical compositions of the present disclosure, including but not limited to those specified in the examples, are useful for treating dilutional coagulopathy of severe blood loss and bleeding in a subject. For example, the pharmaceutical compositions of the present disclosure comprising smaller and more stable SAMS (average diameter of 1-120 nm), NaCl (102 mM), Na (L) lactate (35 mM), L-histidine (50 mM or less) made with egg phospholipid or egg lecithin beyond 2% (w/v) including in an amount of 6% (w/v) or higher, may be used to treat the dilutional coagulopathy of severe blood loss. The pharmaceutical compositions of the present disclosure comprising stabilized SAMS may be combined with plasma, prothrombin complex concentrate or factor VII to produce fluid that has the volume replacement and nitric oxide absorbing capability and that can also activate clotting mechanisms.

The pharmaceutical compositions of the present disclosure, including but not limited to those specified in the examples, are useful for treating lost volume in childhood diseases that cause hypovolemia. For example, the pharmaceutical compositions of the present disclosure comprising smaller and more stable SAMS (average diameter of 1-120 nm), NaCl (102 mM), Na (L) lactate (35 mM), L-histidine (50 mM or less) made with egg phospholipid or egg lecithin beyond 2% (w/v) including in an amount of 6% (w/v) or higher, may be used to replace lost volume in childhood diseases that cause hypovolemia. There are numerous diseases that occur in childhood that can cause severe hypovolemia through the loss of body fluids through vomiting or diarrhea, perspiration. or refusal to take fluids. Specific examples include gastroenteritis and dengue fever.

The pharmaceutical compositions of the present disclosure, including but not limited to those specified in the examples, are useful for treating traumatic brain injury. For example, the pharmaceutical compositions of the present disclosure comprising smaller and more stable SAMS (average diameter of 1-120 nm), NaCl (102 mM), Na (L) lactate (35 mM), L-histidine (50 mM or less) made with egg phospholipid or egg lecithin beyond 2% (w/v) including in an amount of 6% (w/v) or higher, may be used to treat traumatic brain injury. The anti-inflammatory properties of the SAMS can be used to treat traumatic brain injury. In this instance, an oil with greater anti-inflammatory properties than soybean oil may be preferred. Examples are oils that have higher percentage content of omega-3 fatty acids such as oil from fish, algae, or chia beans.

In some embodiments, the pharmaceutical compositions may comprise the following components wherein all % is w/v: algae oil 20%, egg lecithin 12%, NaCl 102 mM, Na (L) lactate, Histidine 10 mM, Cyclosporine A 1×10⁻⁶M, Vitamin E 1 mM, water, pH=7-7.5. Blood is exchanged with the aforementioned formulation ml/ml. Generally, a minimum of 250 ml and a maximum of 5 liters or total blood volume are to be utilized. The formulation is maintained in the circulation for approximately 6 hours. Then the blood is exchanged back ml/ml. If the subject is hypovolemic during or after the second exchange, then hypovolemia can be alleviated with the above-mentioned formulation. The results are monitored with intracranial pressure (ICP) monitor. If the ICP is greater than 20 after treatment, then a decompressive craniectomy is performed. Additionally, intravascular volume in monitored with a device that measures stroke volume index.

The pharmaceutical compositions of the present disclosure, including but not limited to those specified in the examples, are useful for treating burns in a subject. For example, the pharmaceutical compositions of the present disclosure comprising smaller and more stable SAMS (average diameter of 90-120 nm), NaCl (102 mM), Na (L) lactate (35 mM), L-histidine (10 mM or less) made with egg phospholipid or egg lecithin beyond 2% (w/v) including in an amount of 6% (w/v) or higher, may be used to treat burns. Oils similar to those used to treat traumatic brain injury as detailed above, can be useful when infused intravascularly or topically because of their anti-inflammatory properties.

The pharmaceutical compositions of the present disclosure, including but not limited to those specified in the examples, are useful for restoring normal and or a viable cardiac rhythm to the heart when the heart has gone into cardiac arrest or into a life-threatening rhythm by infusion intra-arterially via an artery such as the femoral artery in a subject. For example, the use of SAMS made with egg phospholipid or egg lecithin beyond 2% (w/v) including in an amount of 6% (w/v) or higher results to restore normal and or a viable cardiac rhythm to the heart when the heart has gone into cardiac arrest or into a life-threatening rhythm by infusion intra-arterially via an artery such as the femoral artery. Intra-arterial infusion achieves perfusion of the heart, brain and lungs more effectively than intravenous infusion. If the intra-arterial route is unavailable or inaccessible hen intravenous infusion will also be beneficial. Any reason for the cardiac arrest or arrhythmia would benefit from this approach such as hypovolemia, vasodilation, hypervolemia, electrolyte imbalance or oxygen deficiency. The small micelles or liposomes may be loaded with oxygen

### 6. Diagnosis of Disease

Another aspect of the present disclosure relates to a method for diagnosis of disease in a human or animal subject. For purposes of example only, hydrophobic gas molecules are small enough to pass through the polar head groups that comprise the outer layer of the SAMS of the present disclosure. The polar gases would favor being in the hydrophobic inner space of the micelle just as we have documented with oxygen and nitric oxide. The same would apply to any hydrophobic liquid such as an oil, any amphiphilic molecule and any carrier that can carry a hydrophobic liquid such as an oil and allow gases to pass to the oil. A gas spectrum could also be a diagnostic in which all of the gases detectable by mass spectroscopy or another technique would be assayed and their relative and absolute concentrations would be determined. Other hydrophobic gases and substances could also prove to be useful for diagnosing a disease.

For instance, the pharmaceutical compositions of the present disclosure comprising smaller and more stable SAMS (average diameter of 1-120 nm), NaCl (102 mM), Na (L) lactate (35 mM), L-histidine (1 mM or less or alternatively, no histidine) made with egg phospholipid or egg lecithin beyond 2% (w/v) including in an amount of 6% (w/v) or higher, may be used for diagnosis of diseases. The pharmaceutical compositions comprising SAMS may be infused into the blood stream or into the lymphatic or cerebrospinal fluid system where because of their partition coefficient they can absorb molecules of diagnostic value. The SAMS may also bind to molecules, viruses, bacteria, prions or cancer cells or other living organisms of diagnostic value. Examples of such molecules are endogenously produced carbon monoxide or nitric oxide. Other examples are viruses, prions or cancer cells. After binding or absorbing these entities the SAMS can be removed from the body and analyzed for the diagnostic molecules or organisms that are associated with them.

In some embodiments, the pharmaceutical compositions of the present disclosure can be used for diagnosing a disease in a human or animal subject, and may comprise the following components wherein all % is w/v: soybean oil 20%, egg lecithin 12%, NaCl 102 mM, Na (L) lactate 35 mM, water, pH=7-7.5. Alternatively, the pharmaceutical compositions of the present disclosure to be used for diagnosing a disease in a human or animal subject, may comprise the following components wherein all % is w/v: soybean oil 20%, egg lecithin 12%, NaCl 102 mM, Na (L) lactate 35 mM, water, L-histidine (1 mM or less), pH=7-7.5.

Another aspect of the present disclosure relates to a method for diagnosing diseases in a human or animal subject. For purposes of example only, the method may comprise the following steps: to establish a baseline, H₂S content of the emulsion is determined using mass spectroscopy. A baseline gas spectrum could also be determined. Next at least 500 ml of blood is removed and replaced with an equal volume of emulsion. Ten minutes after the exchange, 10 ml of blood is removed into a vacuum tube and sealed with a diaphragm that allows a needle to be placed through it. The blood is centrifuged at high g force for example 100,000-2,000,000×g. This results in blood in a bottom layer and lipid in a top layer. A sample of the lipid layer is placed into the mass spectrometer to determine the H₂S content or the content of a spectrum of gases such as oxygen, carbon monoxide and nitric oxide. The gas content is compared to the baseline and a diagnosis is made.

### Examples

The following non-limiting examples are put forth to provide those of ordinary skill in the art with a description of methods for the present disclosure, and is not intended to limit the scope of the invention. Efforts have been made to ensure accuracy with respect to numbers used (e.g., amounts, temperature, etc.), but some experimental error and deviation should be accounted for, as one of skill in the art would appreciate. Unless indicated otherwise, parts are parts by weight, molecular weight is weight average molecular weight, temperature is in degrees Centigrade, and pressure is at or near atmospheric.

### Example 1: Stability of SAMS

Two sets of SAMS emulsions were prepared. Emulsion 1 contains 20% w/v soybean oil, 12% w/v egg phospholipids, NaCl 102 mM, Na (L) lactate 35 mM, (L) histidine 0.1 mM with an initial pH of 7.4 adjusted with NaOH. Emulsion 2 contains 30% soybean oil, 12% egg phospholipids, NaCl 102 mM, Na (L) lactate 35 mM, (L) histidine 0.1 mM with an initial pH of 7.4 adjusted with NaOH. The SAMS emulsions were produced with a high pressure homogenizer. SAMS size and zeta potential were measured with a Malvern Zetasizer, Model: Nano ZS.

Below are their zeta potentials:

Emulsion 1 18.40 and −18.26, mean=−18.26 (n=2)

Emulsion 2 −19.57 and −14.85, mean=−17.21 (n=2)

SAMS in Emulsion 1 have an initial average diameter of 91.25 nm, and an average diameter of 92.79 nm at 66 days at room temperature.

SAMS in Emulsion 2 have an initial average diameter of 94.76 nm, and an average diameter of 97.42 nm at 66 days at room temperature.

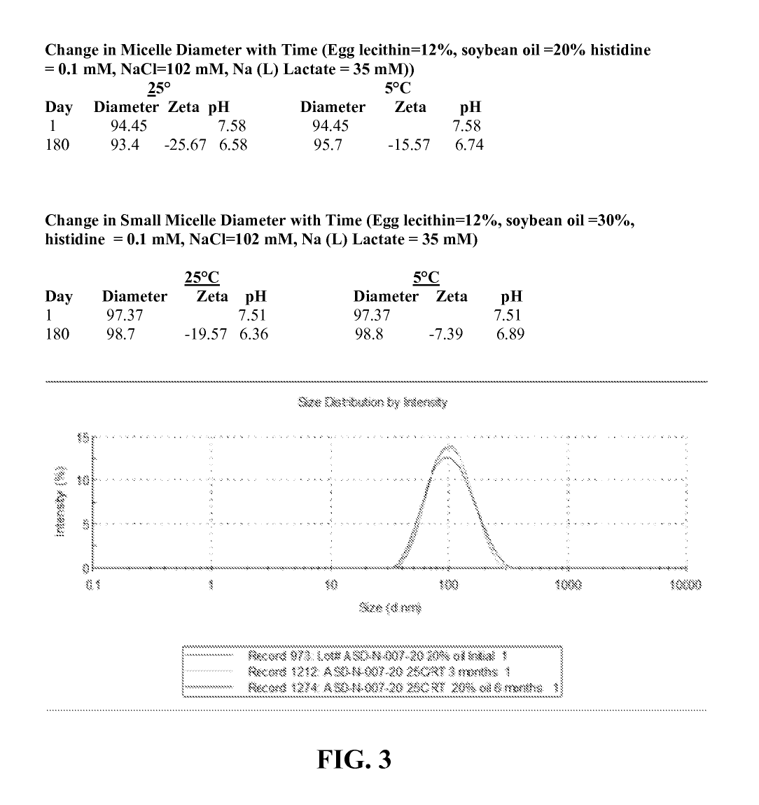

In addition, the SAMs of the present disclosure were stable up to at least 6 months, as shown in FIG. 3.

This stability is unexpected given the low zeta potential of Emulsion 1 or 2 is in the unstable range (i.e., with an absolute value less than 30) according to the Table below:

| Zeta potential [mV] | Stability behavior of the colloid |
|---|---|
| from 0 to ±5, | Rapid coagulation or flocculation |
| from ±10 to ±30 | Incipient instability |
| from ±30 to ±40 | Moderate stability |
| from ±40 to ±60 | Good stability |
| more than ±61 | Excellent stability |

Thus, the pharmaceutical compositions comprising smaller and more stable SAMS (average diameter of 90-120 nm), NaCl (102 mM), Na (L) lactate (35 mM), L-histidine (1 mM or less) made with egg phospholipid or egg lecithin at 2% (w/v) or greater are unexpectedly stable despite having a low zeta potential that predicts instability.

### Example 2: Relationship Between SAMS Size and the Amount of Egg Phospholipid Used for Emulsification

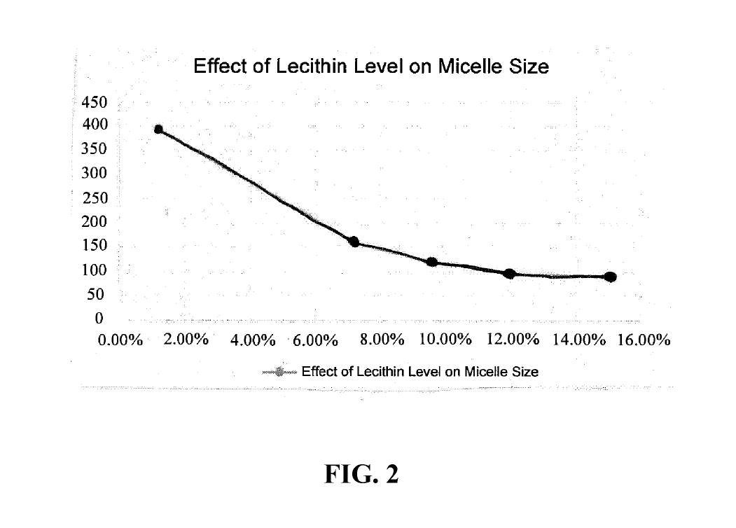

Aqueous mixtures of 20% highly refined soybean oil and various concentrations (w/v) of egg phospholipids were homogenized until the SAMS size stopped decreasing. The resultant SAMS diameter is plotted on the Y axis and the emulsifier (egg phospholipid or egg lecithin) concentration (w/v) is on the X axis of FIG. 2. The decrease in SAMS diameter with respect to emulsifier concentration was insignificant when the amount of the emulsifier is above 12%.

Similar behavior would be expected from SAMS or other configurations of phospholipids in which there are separate hydrophobic and hydrophilic zones. Y axis=Micelle or Liposome average diameter in nm measured using a Malvern Zetasizer model: Nano ZS); X axis=Lecithin=egg lecithin (Lipoid E 80, Ludwigshafen, Germany, contains 80% phosphatidylcholine).

Presently, after mixing the lipid and aqueous phases of such lipid emulsions, one of skill would typically have to use it within 24 hours. With the discovery of the present disclosure, it is possible to premix the two phases and leave it in storage to be used later. Premixing can eliminate the need a significant amount of pharmacy resources that go into providing the TPN/TPA. It can also reduce the possibility of error in mixing and contamination. If the SAMS are unstable, they can coalesce and increase in size, which can block blood flow in capillaries. In addition, they will be more readily taken up by the reticuloendothelial system. The stable, small SAMS of the present disclosure can pass more easily through the liver and splenic sinusoids. This in turn can reduce the adverse effects of SAMS trapping in the sinusoids with loss of bacterial sequestration or other possible consequences.

Such stable, small SAMS have not been used for hyperalimentation. The significantly enhanced SAMS stability of the present disclosure allows electrolytes, amino acids, glucose and other components of total parenteral nutrition to be mixed with the SAMS and stored for 6 months or more. The SAMS currently used in available products for hyperalimentation have diameter of approximately 300 nm. Thus, current SAMS must be mixed with other components on the day of use.

### Example 3: Use of SAMS Made with Egg Phospholipid or Egg Lecithin at 2% (w/v) or Greater to Raise the Blood Pressure of an Animal or Human Subject

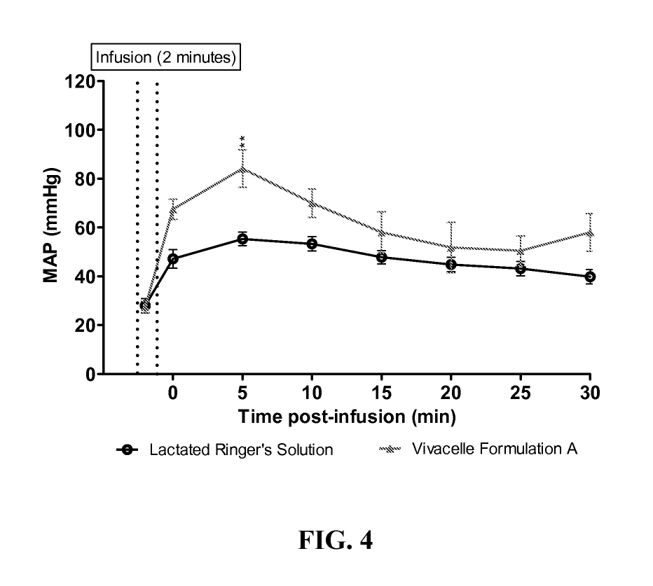

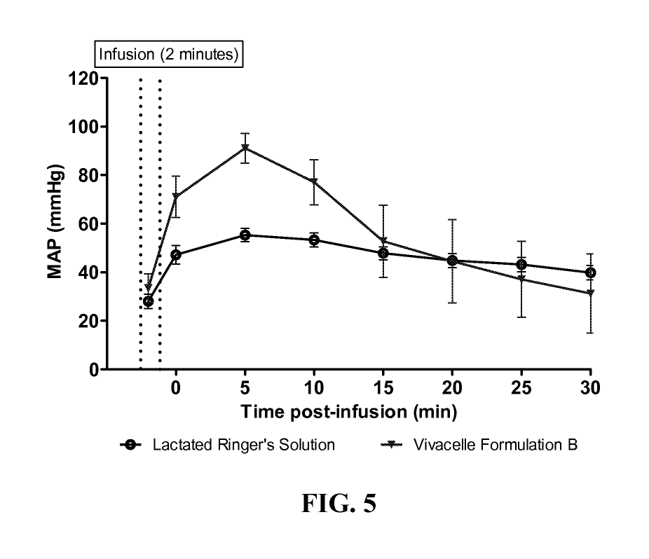

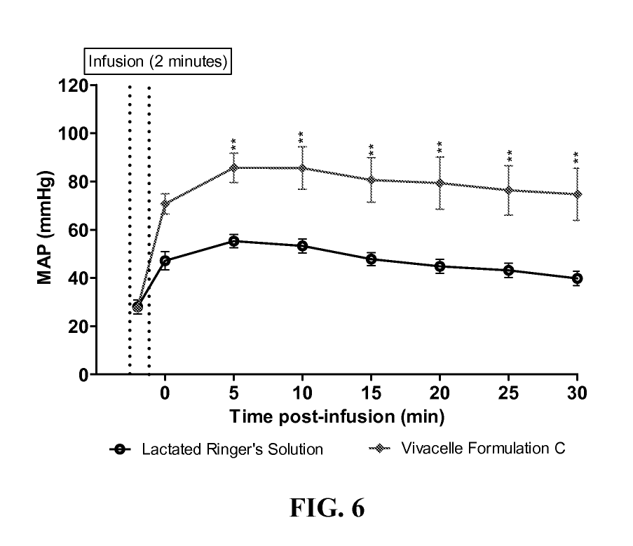

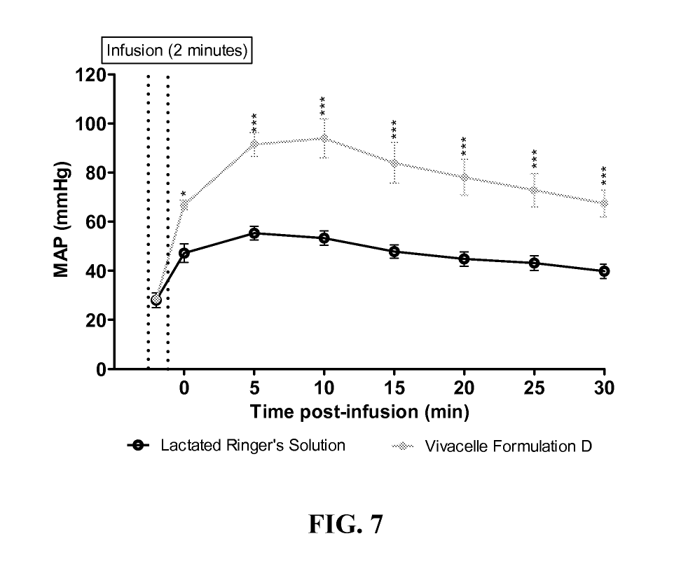

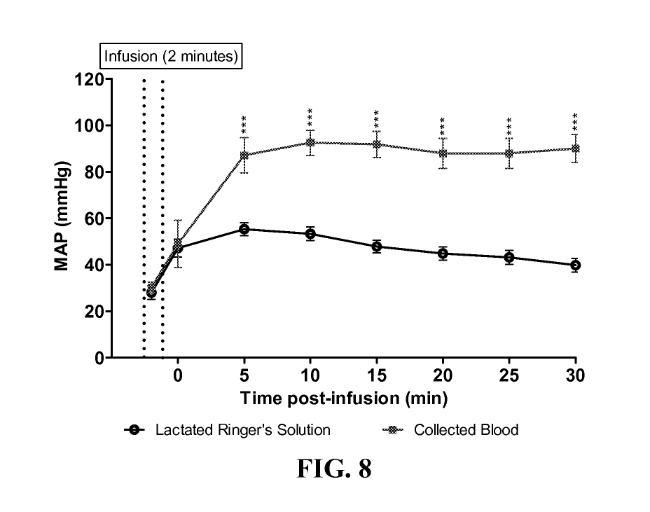

Rats were anesthetized with isoflurane and the femoral artery and vein were cannulated. Next enough blood was removed such that the blood pressure was a mean of 25-30 mmHg. The blood pressure was maintained at this level for one hour by withdrawing or infusing blood as needed. After one hour of shock the rats were infused with one of the following fluids (1) Ringers lactate (commercially obtained), (2) shed blood (3) Formulation A comprising SAMS having an average diameter of 300 nm, soybean oil 20% (w/v); (4) Formulation B comprising SAMS having an average diameter of 300 nm, soybean oil 30% (w/v); (5) Formulation C comprising SAMS having an average diameter of SAMS diameter of 97 nm, soybean oil 20% (w/v) and (6) Formulation D comprising average SAMS diameter of 97 nm, soybean oil and 30% w/v. Formulations A, B, C and D also comprised NaCl (102 mM), Na (L) lactate (35 mM) and L-histidine (1 mM). The results of the formulations are shown in FIGS. 4-8.

Ringer's lactate brought about a relatively small increase in blood pressure. Shed blood increased the blood pressure and maintained it significantly above the blood pressure reached after Ringer's lactate administration, and remained that way for the entire 30 minute observation period. Infusion of Formulations A and B caused an initial, transient increase in the blood pressure, but after 15 minutes, the blood pressure decreased to the level of those rats infused with Ringer's lactate. There was no significant difference between Formulations A and B. In contrast, infusion of Formulations C and D significantly increased the blood pressure throughout the 30 minute observation period.

The expectation was for the blood pressure response to increase with an increase in oil concentration, and this did not occur. Instead, surprisingly, an unexpected result of an increase in blood pressure response with a decrease in the average SAMS diameter was obtained. It is also possible that a reduction in the size of the SAMS contributed to the greater efficacy that we observed.

### Example 4: Use of Small SAMS Made with Egg Phospholipid or Egg Lecithin at 2% (w/v) or Greater for Plasma Exchange in an Animal or Human Subject

The pharmaceutical compositions of the present disclosure comprising smaller and more stable SAMS (average diameter of 90-120 nm), NaCl (102 mM), Na (L) lactate (35 mM), L-histidine (1 mM or less) made with egg phospholipid or egg lecithin beyond 2% (w/v) including in an amount of 6% (w/v) or higher, may be used for exchange transfusion

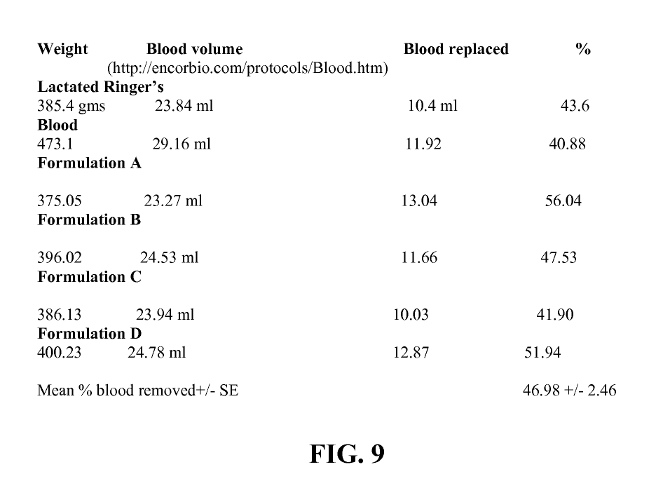

Rats were anesthetized and subjected to severe hemorrhagic shock. As disclosed in FIG. 9, enough blood was removed from the rats until the blood pressure was a mean of 25-30 mmHg. Shed blood was removed or infused as needed to maintain this pressure for one hour. The mean % blood removed+/−SE was 46.98+/−2.46. At the end of 60 minutes at a mean pressure of 25-30 mmHg, test infusate, i.e., SAMS that had a diameter of 300 nm or more stable SAMS with a diameter of 97 nm, were infused over 2 minutes via the femoral vein in a volume equal to the volume of blood that had been removed. In the case of blood, the shed blood was infused. The resulting blood pressure was followed for 30 minutes. The ability of the various test infusates were compared to the effect of Ringer's lactate and observed for 30 minutes. SAMS at 300 nm i.e., Formulations A and B, were minimally better than Ringers lactate. Formulations C and D were significantly better than Ringer's lactate and not statistically different from blood in the 30 minute observation period. It was found that there was no significant difference based on the concentration of soybean oil, and the smaller SAMS produced a marked improvement in the blood pressure response.

The stability of the SAMS of the present disclosure appear to increase the time the SAMS resides in the intravascular space, thereby appearing to be suitable for use in exchange transfusion. This observation can be extended to all of the other applications listed above, in which a more stable formulation will enhance and prolong the efficacy of the formulation. Thus, the SAMS of the present disclosure could also be used to replace a patient's blood, lymph or cerebrospinal fluid in order to remove toxic molecules or bacteria, viruses, cells or prions or other living pathogens from the bloodstream, replace circulating cancer cells and perfuse organs that are much more resistant to immunological attack.

What is claimed is:

1. A pharmaceutical composition comprising:
(a) self-assembling amphiphilic molecular structures (SAMS) with an average diameter of 120 nm or less, wherein the SAMS comprise egg phospholipid in an amount greater than 2% (w/v) of the pharmaceutical composition, and wherein the SAMS have a zeta potential of less than 30 mV; and
(b) from 90 mM to 130 mM of NaCl, from 10 mM to 100 mM of Na (L) lactate, and 10 mM or less of L-histidine.

2. The pharmaceutical composition of claim 1, wherein the pharmaceutical composition comprises:
(a) SAMS with an average diameter of from 90 to 120 nm, wherein the SAMS comprise egg phospholipid in an amount greater than 2% (w/v) of the pharmaceutical composition, and wherein the SAMS have a zeta potential of less than 30 mV; and
(b) 102 mM of NaCl, 35 mM of Na (L) lactate, and 10 mM or less of L-histidine.

3. The pharmaceutical composition of claim 1, wherein the L-histidine concentration is 1 mM or less.

4. The pharmaceutical composition of claim 1, wherein the SAMS further comprise a lipid component in an amount of from 20% to 30% (w/v) of the pharmaceutical composition.

5. The pharmaceutical composition of claim 1, wherein the lipid component is an oil, and wherein the oil is soybean oil, chia bean oil, algae oil, pumpkin oil, flaxseed oil, fish oil or a combination thereof.

6. A pharmaceutical composition for Total Parenteral Nutrition (TPN) or Total Parenteral Alimentation (TPA) comprising:
(a) a pharmaceutically acceptable carrier, wherein the pharmaceutically acceptable carrier is water;
(b) a lipid component in an amount of from 5% to 70% (w/v) of the pharmaceutical composition; and
(c) an amphiphilic emulsifier in an amount of 6% or higher (w/v) of the pharmaceutical composition,
wherein the lipid component and the amphiphilic emulsifier form SAMS in the water carrier, wherein the SAMS have an average diameter of 120 nm or less, wherein the SAMS have a zeta potential of less than 30 mV, and wherein the pharmaceutical composition is free of hemoglobin and fluorocarbon.

7. The pharmaceutical composition of claim 6, wherein the lipid component is in an amount of from 20% to 30% (w/v) of the total pharmaceutical composition, and wherein the lipid component comprises an oil, wherein the oil is soybean oil, chia bean oil, algae oil, pumpkin oil, flaxseed oil, fish oil or a combination thereof.

8. A method for treating conditions of intravascular volume loss in a human or animal subject in need thereof, the method comprising:
administering to said subject a therapeutically effective amount of a pharmaceutical composition comprising SAMS with an average diameter of from 90 to 120 nm, 102 mM of NaCl, 35 mM of Na (L) lactate, and 10 mM or less of L-histidine,
wherein the SAMS comprise egg phospholipid in an amount greater than 2% (w/v) of the pharmaceutical composition, and a zeta potential of less than 30 mV.

9. A method for treating excessive production of nitric oxide in a human or animal subject in need thereof, the method comprising:
administering to said subject a therapeutically effective amount of a pharmaceutical composition comprising SAMS with an average diameter of from 90 to 120 nm, 102 mM of NaCl, 35 mM of Na (L) lactate, and 10 mM or less of L-histidine,
wherein the SAMS comprise egg phospholipid in an amount greater than 2% (w/v) of the pharmaceutical composition, and have a zeta potential of less than 30 mV.

10. The method of claim 9, wherein the SAMS further comprise a lipid component in an amount of from 20% to 30% (w/v) of the pharmaceutical composition, and wherein the lipid component is soybean oil, chia bean oil, algae oil, pumpkin oil, flaxseed oil, fish oil or a combination thereof.

11. A method for increasing the blood pressure in a human or animal subject in need thereof, the method comprising:
administering to said subject a therapeutically effective amount of a pharmaceutical composition comprising SAMS with an average diameter of from 90 to 120 nm, 102 mM of NaCl, 35 mM of Na (L) lactate, and 10 mM or less of L-histidine,
wherein the SAMS comprise egg phospholipid in an amount greater than 2% (w/v) of the pharmaceutical composition, and have a zeta potential of less than 30 mV.

12. The method of claim 11, wherein the SAMS further comprise a lipid component in an amount of from 20% to 30% (w/v) of the pharmaceutical composition, and wherein the lipid component is soybean oil, chia bean oil, algae oil, pumpkin oil, flaxseed oil, fish oil or a combination thereof.

13. A method for treating traumatic brain injury in a human or animal subject in need thereof, the method comprising:
administering to said subject a therapeutically effective amount of a pharmaceutical composition comprising SAMS with an average diameter of from 90 to 120 nm, 102 mM of NaCl, 35 mM of Na (L) lactate, and 10 mM or less of L-histidine,
wherein the SAMS comprise egg phospholipid in an amount greater than 2% (w/v) of the pharmaceutical composition, wherein the SAMS further comprise a lipid component in an amount of from 20% to 30% (w/v) of the pharmaceutical composition, wherein the lipid component is soybean oil, chia bean oil, algae oil, pumpkin oil, flaxseed oil, fish oil or a combination thereof, and wherein the SAMS have a zeta potential of less than 30 mV.

14. A method of conducting renal dialysis in a human or animal subject in need thereof, the method comprising:
administering to said subject a therapeutically effective amount of a pharmaceutical composition comprising SAMS with an average diameter of from 90 to 120 nm, 102 mM of NaCl, 35 mM of Na (L) lactate, and 10 mM or less of L-histidine,
wherein the SAMS comprise egg phospholipid in an amount greater than 2% (w/v) of the pharmaceutical composition, wherein the SAMS further comprise a lipid component in an amount of from 20% to 30% (w/v) of the pharmaceutical composition, wherein the lipid component is soybean oil, chia bean oil, algae oil, pumpkin oil, flaxseed oil, fish oil or a combination thereof, and wherein the SAMS have a zeta potential of less than 30 mV.

15. A method for conducting an exchange transfusion to replace plasma or whole blood that contains harmful mediators, prions, viruses, bacteria, fungi, chemical or biological agents of warfare, cancer cells or other bloodstream born agents in a human or animal subject in need thereof, the method comprising:
administering to said subject a therapeutically effective amount of a pharmaceutical composition comprising SAMS with an average diameter of from 90 to 120 nm, 102 mM of NaCl, 35 mM of Na (L) lactate, and 10 mM or less of L-histidine,
wherein the SAMS comprise egg phospholipid in an amount greater than 2% (w/v) of the pharmaceutical composition, wherein the SAMS further comprise a lipid component in an amount of from 20% to 30% (w/v) of the pharmaceutical composition, wherein the lipid component is soybean oil, chia bean oil, algae oil, pumpkin oil, flaxseed oil, fish oil or a combination thereof, and wherein the SAMS have a zeta potential of less than 30 mV.

16. A method for diagnosing diseases in a human or animal subject in need thereof, the method comprising:
administering to said subject a therapeutically effective amount of a pharmaceutical composition comprising SAMS with an average diameter of from 90 to 120 nm, 102 mM of NaCl, 35 mM of Na (L) lactate, and 10 mM or less of L-histidine,
wherein the SAMS comprise egg phospholipid in an amount greater than 2% (w/v) of the pharmaceutical composition, wherein the SAMS further comprise a lipid component in an amount of from 20% to 30% (w/v) of the pharmaceutical composition, wherein the lipid component is soybean oil, chia bean oil, algae oil, pumpkin oil, flaxseed oil, fish oil or a combination thereof, and wherein the SAMS have a zeta potential of less than 30 mV.

17. A method for treating septic shock and irreversible shock in a human or animal subject in need thereof, the method comprising:
administering to said subject a therapeutically effective amount of a pharmaceutical composition comprising SAMS with an average diameter of from 90 to 120 nm, 102 mM of NaCl, 35 mM of Na (L) lactate, and 10 mM or less of L-histidine,
wherein the SAMS comprise egg phospholipid in an amount greater than 2% (w/v) of the pharmaceutical composition, wherein the SAMS further comprise a lipid component in an amount of from 20% to 30% (w/v) of the pharmaceutical composition, wherein the lipid component is soybean oil, chia bean oil, algae oil, pumpkin oil, flaxseed oil, fish oil or a combination thereof, and wherein the SAMS have a zeta potential of less than 30 mV.

18. A method for conducting Extracorporeal Membrane Oxygenation (ECMO) in a human or animal subject in need thereof, the method comprising:
administering to said subject a therapeutically effective amount of a pharmaceutical composition comprising SAMS with an average diameter of from 90 to 120 nm, 102 mM of NaCl, 35 mM of Na (L) lactate, and 10 mM or less of L-histidine,
wherein the SAMS comprise egg phospholipid in an amount greater than 2% (w/v) of the pharmaceutical composition, wherein the SAMS further comprise a lipid component in an amount of from 20% to 30% (w/v) of the pharmaceutical composition, wherein the lipid component is soybean oil, chia bean oil, algae oil, pumpkin oil, flaxseed oil, fish oil or a combination thereof, and wherein the SAMS have a zeta potential of less than 30 mV.

19. A method for opening constricted or blocked blood vessels in a human or animal subject in need thereof, the method comprising:
administering to said subject a therapeutically effective amount of a pharmaceutical composition comprising SAMS with an average diameter of from 90 to 120 nm, 102 mM of NaCl, 35 mM of Na (L) lactate, and 10 mM or less of L-histidine,
wherein the SAMS comprise egg phospholipid in an amount greater than 2% (w/v) of the pharmaceutical composition, wherein the SAMS further comprise a lipid component in an amount of from 20% to 30% (w/v) of the pharmaceutical composition, wherein the lipid component is soybean oil, chia bean oil, algae oil, pumpkin oil, flaxseed oil, fish oil or a combination thereof, and wherein the SAMS have a zeta potential of less than 30 mV.

20. A method for treating dilutional coagulopathy of severe blood loss and bleeding in a human or animal subject in need thereof, the method comprising:
administering to said subject a therapeutically effective amount of a pharmaceutical composition comprising SAMS with an average diameter of from 90 to 120 nm, 102 mM of NaCl, 35 mM of Na (L) lactate, and 10 mM or less of L-histidine,
wherein the SAMS comprise egg phospholipid in an amount greater than 2% (w/v) of the pharmaceutical composition, wherein the SAMS further comprise a lipid component in an amount of from 20% to 30% (w/v) of the pharmaceutical composition, wherein the lipid component is soybean oil, chia bean oil, algae oil, pumpkin oil, flaxseed oil, fish oil or a combination thereof, and wherein the SAMS have a zeta potential of less than 30 mV.

21. A method for transporting therapeutic gases such as oxygen, nitric oxide, hydrogen sulfide, xenon, argon or carbon monoxide in a human or animal subject in need thereof, the method comprising:
administering to said subject a therapeutically effective amount of a pharmaceutical composition comprising SAMS with an average diameter of from 90 to 120 nm, 102 mM of NaCl, 35 mM of Na (L) lactate, and 10 mM or less of L-histidine,
wherein the SAMS comprise egg phospholipid in an amount greater than 2% (w/v) of the pharmaceutical composition, wherein the SAMS further comprise a lipid component in an amount of from 20% to 30% (w/v) of the pharmaceutical composition, wherein the lipid component is soybean oil, chia bean oil, algae oil, pumpkin oil, flaxseed oil, fish oil or a combination thereof, and wherein the SAMS have a zeta potential of less than 30 mV.

22. A method for treating burns in a human or animal subject in need thereof, the method comprising:
administering to said subject a therapeutically effective amount of a pharmaceutical composition comprising SAMS with an average diameter of from 90 to 120 nm, 102 mM of NaCl, 35 mM of Na (L) lactate, and 10 mM or less of L-histidine,
wherein the SAMS comprise egg phospholipid in an amount greater than 2% (w/v) of the pharmaceutical composition, wherein the SAMS further comprise a lipid component in an amount of from 20% to 30% (w/v) of the pharmaceutical composition, wherein the lipid component is soybean oil, chia bean oil, algae oil, pumpkin oil, flaxseed oil, fish oil or a combination thereof, and wherein the SAMS have a zeta potential of less than 30 mV.
